(19) United States
(12) Patent Application Publication
(10) Pub. No.: US 2009/0118220 A1
(43) Pub. Date: May 7, 2009

(54) SUBSTITUTED ADENINES AND THE USES THEREOF

(75) Inventors: Marta Cavero-Tomas, Wayland, MA (US); Jenna Harang, Waltham, MA (US); Hoan Khai Huynh, Waltham, MA (US); Haihong Ni, Waltham, MA (US); Suzanne Stokes, Waltham, MA (US)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 12/296,033

(22) PCT Filed: Apr. 2, 2007
(86) PCT No.: PCT/GB07/01207

§ 371 Date: Oct. 3, 2008

Related U.S. Application Data
(60) Provisional application No. 60/744,151, filed on Apr. 3, 2006.

(51) Int. Cl. A61K 31/7076; C07H 19/16; A61P 31/04

(52) U.S. Cl. 514/45; 536/27.7

(57) ABSTRACT

This invention relates to compounds of Formula I:

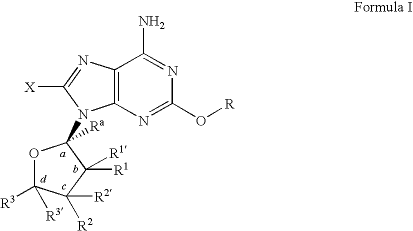

and their use in the treatment of bacterial infections.

### BACKGROUND OF THE INVENTION

The international microbiological community continues to express serious concern that the evolution of antibacterial resistance could result in bacterial strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity.

Gram-positive pathogens, for example staphylococci, enterococci, streptococci and mycobacteria, are particularly important because of the development of resistant strains that are both difficult to treat and difficult to eradicate from the hospital environment once established. Examples of such strains are methicillin resistant Staphylococcus aureus (MRSA), methicillin resistant coagulase-negative staphylococci (MRCNS), penicillin resistant Streptococcus pneumoniae and multiple resistant Enterococcus faecium. The preferred clinically effective antibiotic of last resort for treatment of such resistant Gram-positive pathogens is vancomycin. Vancomycin is a glycopeptide and is associated with various toxicities, including nephrotoxicity. Furthermore, and most importantly, antibacterial resistance to vancomycin and other glycopeptides is also appearing. This resistance is increasing at a steady rate rendering these agents less effective in the treatment of Gram-positive pathogens. There is also increasing resistance to agents such as β-lactams, quinolones and macrolides used for the treatment of upper respiratory tract infections caused by Gram-negative strains including H. influenzae and M. catarrhalis. Consequently, in order to overcome the threat of widespread multi-drug resistant organisms, there is an on-going need to develop new antibacterials, particularly those with either a novel mechanism of action and/or containing new pharmacophoric groups.

Deoxyribonucleic acid (DNA) ligases catalyze the formation of a phosphodiester linkage at single-strand breaks between adjacent 3′-OH and 5′-phosphate termini in double-stranded DNA (Lehman 1974. Science 186: 790-797). This activity plays an indispensable role in DNA replication where it joins Okazaki fragments. DNA ligase also plays a role in repair of damaged DNA and in recombination (Wilkinson 2001. Molecular Microbiology 40: 1241-1248). An early report describing conditional lethal mutations in the DNA ligase gene (ligA) of Escherichia coli supported the essentiality of this enzyme (Dermody et al. 1979. Journal of Bacteriology 139: 701-704). This was followed by the isolation and characterization of DNA ligase temperature-sensitive or knockout mutants of Salmonella typhimurium, Bacillus subtilis, and Staphylococcus aureus (Park et al. 1989. Journal of Bacteriology 171: 2173-2180, Kaczmarek et al. 2001. Journal of Bacteriology 183: 3016-3024, Petit and Ehrlich. 2000. Nucleic Acids Research 28: 4642-4648). In all species, DNA ligase was shown to be essential.

The DNA ligase family can be divided into two classes: those requiring ATP for adenylation (eukaryotic cells, viruses and bacteriophages), and those requiring NAD⁺ (nicotinamide adenine dinucleotide) for adenylation, which include all known bacterial DNA ligases (Wilkinson 2001, supra). Eukaryotic, bacteriophage, and viral DNA ligases show little sequence homology to DNA ligases from prokaryotes, apart from a conserved KXDG motif located within the central cofactor-binding core of the enzyme. Amino acid sequence comparisons clearly show that NAD⁺-dependent ligases are phylogenically unrelated to the ATP-dependent DNA ligases. The apparent lack of similarity between the DNA ligases of bacteria and those of higher organisms suggests that bacterial DNA ligase is a good target for developing new antibacterials.

In 2003, Brötz-Oesterhelt et al. (Journal of Biological Chemistry 278:39435-39442) reported pyridochromanones as the first example of a selectively potent class of bacterial DNA ligase inhibitors whose mode of action was confirmed. This publication demonstrated proof-of-principle validation of LigA as an antibacterial target.

As a result, there is need for compounds that inhibit LigA and thus that are useful as antibacterial agents.

### SUMMARY OF THE INVENTION

These and other needs are met by the present invention which is directed to a compound of formula I

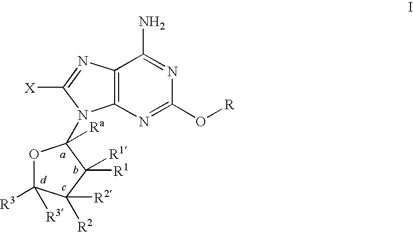

or a pharmaceutically acceptable salt thereof, wherein:

X is hydrogen, halo, NR⁸R⁹, azido, cyano, isocyano, hydroxy, C₁₋₆alkyl, C₂₋₆alkenyl, C₂₋₆alkynyl, aryl, C₃₋₈cycloalkyl, C₃₋₈cycloalkenyl, heterocyclyl, —OR⁷, —C(O)R⁵, —OC(O)R⁵, S(O)ₚR⁴, —NHC(O)NR⁸R⁹, —N(C₁₋₆alkyl)C(O)NR⁸R⁹, —NHC(O)R⁷, —NHCO₂R⁷, —NHSO₂(R⁴), -amidino i.e. —NHC(NH)NH₂; provided that when X is hydrogen, at least one carbon selected from C-a, C-b, C-c, and C-d is quaternary;

R is selected from C₁₋₈alkyl, C₂₋₈alkenyl, C₂₋₈alkynyl, C₃₋₈carbocyclyl, aryl, and heterocyclyl, any of which may be optionally substituted on one or more carbon atom by R′;

p is independently at each occurrence 0, 1 or 2;

R^{a} is hydrogen, C₁₋₆alkyl, C₂₋₆alkenyl, hydroxy(C₁₋₆alkyl), and cyano, any of which may be optionally substituted on one or more carbon atom by R′;

R¹, R^{1′}, R², R^{2′}, R³, and R^{3′} are each independently selected from hydrogen, hydroxy, cyano, azido, C₁₋₆alkyl, C₃₋₈carbocyclyl, halo, —C(O)R⁵, —OC(O)R⁵, S(O)ₚR⁴, C₂₋₆alkenyl, C₂₋₆alkynyl, heterocyclyl, —OR⁷, NR⁸R⁹, wherein R¹, R^{1′}, R², R^{2′}, R³, and R^{3′} may be optionally substituted on one or more carbon atoms by one or more R; or alternatively,

R¹ and R^{1′}, R² and R^{2′}, or R³ and R^{3′}, taken together with the carbon to which they are attached, form C═O or C═N—O—R⁶, or an optionally substituted 3, 4, 5, 6, or 7-membered ring containing 0, 1, or 2 heteroatoms selected from O, S, NH, or N(C₁₋₆alkyl); or alternatively,

R¹ and R², R² and R³, taken together with the carbons to which they are attached, form an optionally substituted 3, 4, 5, or 6-membered ring containing 0, 1, or 2 heteroatoms selected from O, S, NH, or N(C₁₋₆alkyl);

R⁴ at each occurrence is independently —NR⁸R⁹, C₁₋₆alkyl, C₂₋₆alkenyl, C₁₋₆alkoxy, C₃₋₈cycloalkyl, heterocyclyl, and aryl wherein R⁴ may be optionally substituted on one or more carbon atoms by one or more R′;

R⁵ at each occurrence is independently hydrogen, —NR⁸R⁹, —OR⁷, C₁₋₆alkyl, C₂₋₆alkenyl, C₃₋₈cycloalkyl, C₃₋₈cycloalkenyl, heterocyclyl, and aryl wherein each R⁵ may be optionally substituted on one or more carbon atoms by one or more R′;

R⁶ at each occurrence is independently hydrogen, C₁₋₆alkyl, C₂₋₆alkenyl, C₂₋₆alkynyl, C₃₋₈cycloalkyl, C₃₋₈cycloalkenyl, heterocyclyl and aryl, wherein each R⁶ may be optionally substituted on one or more carbon atoms by one or more R′;

R⁷ at each occurrence is independently hydrogen, C₁₋₆alkyl, C₂₋₆alkenyl, C₃₋₈cycloalkyl, C₃₋₈cycloalkenyl, aryl, S(O)ₚR⁴, and heterocyclyl wherein R⁷ may be optionally substituted on one or more carbon by one or more R′;

R⁸ and R⁹ are each independently selected from hydrogen, C₁₋₆alkyl, C₂₋₆alkenyl, C₂₋₆alkynyl, —OR⁷, C₃₋₈cycloalkyl, C₃₋₈cycloalkenyl, heterocyclyl, and aryl, wherein each R⁸ or R⁹ may be optionally substituted on one or more carbon atoms by one or more R′;

R′ at each occurrence is independently halo, hydroxy, nitro, —NR⁸R⁹, azido, cyano, isocyano, C₁₋₆alkyl, C₂₋₆alkenyl, C₂₋₆alkynyl, aryl, C₃₋₈cycloalkyl, C₃₋₈cycloalkenyl, heterocyclyl, keto(═O), —OR⁷, —C(O)R⁵, —OC(O)R⁵, S(O)ₚR⁴; ═N—O—R⁶, —NHC(O)NR⁸R⁹, —N(C₁₋₆alkyl)C(O)NR⁸R⁹, —NHC(O)R⁷, —NHCO₂R⁷, —NHSO₂(R⁴), -amidino i.e. —NHC(NH)NH₂, wherein each R′ may be optionally substituted on one or more carbon by one or more R″;

R″ at each occurrence is independently halo, azido, cyano, C₁₋₆alkyl, C₂₋₆alkenyl, C₂₋₆alkynyl, aryl, cycloalkyl, cycloalkenyl, heterocyclyl, hydroxy, —OR⁷, —C(O)R⁵, —OC(O)R⁵, S(O)ₚR⁴, —NR⁸R⁹, -amidino i.e. —NHC(NH)NH₂; provided the compound is not 8-amino-2-methoxyadenosine.

In another embodiment, the present invention is directed to a compounds of formula II

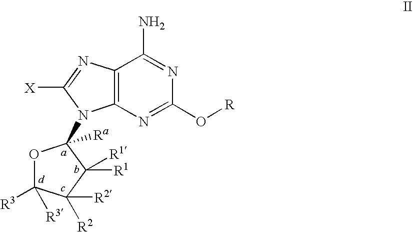

or a pharmaceutically acceptable salt thereof, wherein:

X is hydrogen, halo, NR⁸R⁹, azido, cyano, isocyano, hydroxy, C₁₋₆alkyl, C₂₋₆alkenyl, C₂₋₆alkynyl, aryl, C₃₋₈cycloalkyl, C₃₋₈cycloalkenyl, heterocyclyl, —OR⁷, —C(O)R⁵, —OC(O)R⁵, S(O)ₚR⁴, —NHC(O)NR⁸R⁹, —N(C₁₋₆alkyl)C(O)NR⁸R⁹, —NHC(O)R⁷, —NHCO₂R⁷, —NHSO₂(R⁴), -amidino i.e. —NHC(NH)NH₂; provided that when X is hydrogen, at least one carbon selected from C-a, C-b, C-c, and C-d is quaternary;

R is selected from C₁₋₈alkyl, C₂₋₈alkenyl, C₂₋₈alkynyl, C₃₋₈carbocyclyl, aryl, and heterocyclyl, any of which may be optionally substituted on one or more carbon atom by R′;

p is independently at each occurrence 0, 1 or 2;

R^{a} is hydrogen, C₁₋₆alkyl, C₂₋₆alkenyl, hydroxy(C₁₋₆alkyl), and cyano, any of which may be optionally substituted on one or more carbon atom by R′;

R¹, R^{1′}, R², R^{2′}, R³, and R^{3′} are each independently selected from hydrogen, hydroxy, cyano, azido, C₁₋₆alkyl, C₃₋₈carbocyclyl, halo, —C(O)R⁵, —OC(O)R⁵, S(O)ₚR⁴, C₂₋₆alkenyl, C₂₋₆alkynyl, heterocyclyl, —OR⁷, NR⁸R⁹, wherein R¹, R^{1′}, R², R^{2′}, R³, and R^{3′} may be optionally substituted on one or more carbon atoms by one or more R′; or alternatively,

R¹ and R^{1′}, R² and R^{2′}, or R³ and R^{3′}, taken together with the carbon to which they are attached, form C═O or C═N—O—R⁶, or an optionally substituted 3, 4, 5, 6, or 7-membered ring containing 0, 1, or 2 heteroatoms selected from O, S, NH, or N(C₁₋₆alkyl); or alternatively,

R¹ and R², R² and R³, taken together with the carbons to which they are attached, form an optionally substituted 3, 4, 5, or 6-membered ring containing 0, 1, or 2 heteroatoms selected from O, S, NH, or N(C₁₋₆alkyl);

R⁴ at each occurrence is independently —NR⁸R⁹, C₁₋₆alkyl, C₂₋₆alkenyl, C₁₋₆alkoxy, C₃₋₈cycloalkyl, heterocyclyl, and aryl wherein R⁴ may be optionally substituted on one or more carbon atoms by one or more R;

R⁵ at each occurrence is independently hydrogen, —NR⁸R⁹, —OR⁷, C₁₋₆alkyl, C₂₋₆alkenyl, C₃₋₈cycloalkyl, C₃₋₈cycloalkenyl, heterocyclyl, and aryl wherein each R⁵ may be optionally substituted on one or more carbon atoms by one or more R′;

R⁶ at each occurrence is independently hydrogen, C₁₋₆alkyl, C₂₋₆alkenyl, C₂₋₆alkynyl, C₃₋₈cycloalkyl, C₃₋₈cycloalkenyl, heterocyclyl and aryl, wherein each R⁶ may be optionally substituted on one or more carbon atoms by one or more R′;

R⁷ at each occurrence is independently hydrogen, C₁₋₆alkyl, C₂₋₆alkenyl, C₃₋₈cycloalkyl, C₃₋₈cycloalkenyl, aryl, S(O)ₚR⁴, and heterocyclyl wherein R⁷ may be optionally substituted on one or more carbon by one or more R′;

R⁸ and R⁹ are each independently selected from hydrogen, C₁₋₆alkyl, C₂₋₆alkenyl, C₂₋₆alkynyl, —OR⁷, C₃₋₈cycloalkyl, C₃₋₈cycloalkenyl, heterocyclyl, and aryl, wherein each R⁸ or R⁹ may be optionally substituted on one or more carbon atoms by one or more R′;

R′ at each occurrence is independently halo, hydroxy, nitro, —NR⁸R⁹, azido, cyano, isocyano, C₁₋₆alkyl, C₂₋₆alkenyl, C₂₋₆alkynyl, aryl, C₃₋₈cycloalkyl, C₃₋₈cycloalkenyl, heterocyclyl, keto(═O), —OR⁷, —C(O)R⁵, —OC(O)R⁵, S(O)ₚR⁴; ═N—O—R⁶, —NHC(O)NR⁸R⁹, —N(C₁₋₆alkyl)C(O)NR⁸R⁹, —NHC(O)R⁷, —NHCO₂R⁷, —NHSO₂(R⁴), -amidino i.e. —NHC(NH)NH₂, wherein each R′ may be optionally substituted on one or more carbon by one or more R″;

R″ at each occurrence is independently halo, azido, cyano, C₁₋₆alkyl, C₂₋₆alkenyl, C₂₋₆alkynyl, aryl, cycloalkyl, cycloalkenyl, heterocyclyl, hydroxy, —OR⁷, —C(O)R⁵, —OC(O)R⁵, S(O)ₚR⁴, —NR⁸R⁹, -amidino i.e. —NHC(NH)NH₂;

provided the compound is not 8-amino-2-methoxyadenosine.

The invention further provides compounds of Formula I or Formula II, in free or salt form, e.g., pharmaceutically acceptable salt form, as follows:
1.1 Compounds of Formula I or Formula II, wherein R is selected from a group consisting of C₁₋₈alkyl, C₂₋₈alkenyl, C₂₋₈alkynyl, C₃₋₈carbocyclyl, aryl and heterocyclyl, wherein R is optionally substituted with one or more R′.
1.2 Compounds of Formula I, Formula II or 1.1, wherein R is C₃₋₈carbocyclyl.
1.3 Compounds of Formula I or Formula II, wherein R is cyclopentyl.
1.4 Compounds of Formula I, Formula II or any of 1.1-1.3, wherein R^{a} is selected from a group consisting of hydrogen, C₁₋₆alkyl, C₂₋₆alkenyl, hydroxy(C₁₋₆alkyl), and cyano, wherein R^{a} is optionally substituted with one or more R′.
1.5 Compounds of Formula I, Formula II or any of 1.1-1.4, wherein R^{a} is hydroxymethyl.
1.6 Compounds of Formula I, Formula II or any of 1.1-1.4, wherein R^{a} is hydrogen
1.7 Compounds of Formula I, Formula II or any of 1.1-1.6, wherein X is selected from a group consisting of hydrogen, halo, NR⁸R⁹, azido, cyano, isocyano, hydroxy, C₁₋₆alkyl, C₂₋₆alkenyl, C₂₋₆alkynyl, aryl, C₃₋₈cycloalkyl, C₃₋₈cycloalkenyl, heterocyclyl, —OR⁷, —C(O)R⁵, —OC(O)R⁵, S(O)ₚR⁴, —NHC(O)NR⁸R⁹, —N(C₁₋₆alkyl)C(O)NR⁸R⁹, —NHC(O)R⁷, —NHCO₂R⁷, —NHSO₂(R⁴), -amidino i.e. —NHC(NH)NH₂; provided that when X is hydrogen, at least one carbon selected from C-a, C-b, C-c, and C-d is quaternary.
1.8 Compounds of Formula I, Formula II or any of 1.1-1.7, wherein X is selected from a group consisting of hydrogen, halo and NR⁸R⁹ provided that when X is hydrogen, at least one carbon selected from C-a, C-b, C-c, and C-d is quaternary.
1.9 Compounds of Formula I, Formula II or any of 1.1-1.8, wherein X is bromo, amino, alkylamino (e.g., methylamino), optionally substituted phenylmethylamino (e.g., phenylmethylamino or 4-methoxyphenylmethylamino).
1.10 Compounds of Formula I, Formula II or any of 1.1-1.9, wherein X is hydrogen.
1.11 Compounds of Formula I, Formula II or any of 1.1-1.10, wherein R¹ and R^{1′} are independently selected from a group consisting of hydrogen, hydroxy, cyano, azido, C₁₋₆alkyl, C₃₋₈carbocyclyl, halo, —C(O)R⁵, —OC(O)R⁵, S(O)ₚR⁴, C₂₋₆alkenyl, C₂₋₆alkynyl, heterocyclyl, —OR⁷ and NR⁸R⁹, wherein R¹ and R^{1′} are optionally substituted with one or more R′.
1.12 Compounds of Formula I, Formula II or any of 1.1-1.11, wherein R¹ and R^{1′} are independently selected from a group consisting of hydrogen, hydroxy, hydroxyalkyl or alkenyl.
1.13 Compounds of Formula I, Formula II or any of 1.1-1.12, wherein one of R¹ or R^{1′} is independently selected from a group consisting of hydrogen, hydroxy, hydroxymethyl or ethenyl.
1.14 Compounds of Formula I, Formula II or any of 1.1-1.13, wherein one of R¹ or R^{1′} is hydroxy.
1.15 Compounds of Formula I, Formula II or any of 1.1-1.14, wherein one of R¹ or R^{1′} is hydroxy and the other is hydrogen.
1.16 Compounds of Formula I, Formula II or any of 1.1-1.14, wherein one of R¹ or R^{1′} is hydroxy and said hydroxy group is cis to R^{a}.
1.17 Compounds of Formula I, Formula II or any of 1.1-14, wherein R¹ is hydroxy and said hydroxy group is cis to R^{a} and R^{1′} is hydrogen wherein said hydrogen group is trans to R^{a}.
1.18 Compounds of Formula I, Formula II or any of 1.1-1.14, wherein R¹ is hydroxy wherein said hydroxy group is trans to R^{a} and R^{1′} is hydroxymethyl wherein said hydroxymethyl group is cis to R^{a}.
1.19 Compounds of Formula I, Formula II or any of 1.1-1.14, wherein R¹ is hydroxy wherein said hydroxy group is trans to R^{a} and R^{1′} is ethenyl wherein said ethenyl group is cis to R^{a}.
1.20 Compounds of Formula I, Formula II or any of 1.1-1.19, wherein R² and R^{2′} are independently selected from a group consisting of hydrogen, hydroxy, cyano, cyanoalkyl (e.g., acetonitrile), azido, C₁₋₆alkyl, C₃₋₈carbocyclyl, halo, —C(O)R⁵, —OC(O)R⁵, S(O)ₚR⁴, C₂₋₆alkenyl, C₂₋₆alkynyl, heterocyclyl, —OR⁷ and NR⁸R⁹, wherein R² and R^{2′} are optionally substituted with one or more R′.
1.21 Compounds of Formula I, Formula II or any of 1.1-1.20, wherein R² and R^{2′} are independently selected from a group consisting of hydrogen, hydroxy, hydroxymethyl, trifluoromethyl, ethyl and ethenyl.
1.22 Compounds of Formula I, Formula II or any of 1.1-1.21, wherein one of R² or R^{2′} is hydroxy.
1.23 Compounds of Formula I, Formula II or any of 1.1-1.21, wherein one of R² or R^{2′} is hydroxy and the other is hydrogen.
1.24 Compounds of Formula I, Formula II or any of 1.1-1.22, wherein either R¹ or R^{1′} and either R² or R^{2′} are cis- or trans-diol.
1.25 Compounds of Formula I, Formula II or any of 1.1-1.22, wherein either R¹ or R^{1′} and either R² or R^{2′} are cis-diol and said cis-diol is cis to R^{a}.
1.26 Compounds of Formula I, Formula II or any of 1.1-1.25, wherein R¹ and one of R² or R^{2′} are cis-diol wherein said cis-diol is cis to R^{a} and R^{1′} is hydrogen or trifluoromethyl wherein said hydrogen or trifluoromethyl is trans to R^{a}.
1.27 Compounds of Formula I, Formula II or any of 1.1-1.25, wherein R¹ is hydroxy wherein said hydroxy is trans to R^{a} and R^{1′} is ethyl, ethenyl or hydroxymethyl wherein said ethyl, ethenyl or hydroxymethyl is cis to R^{a}.
1.28 Compounds of Formula I, Formula II or any of 1.1-1.27, wherein R³ and R^{3′} are independently selected from a group consisting of hydrogen, hydroxy, cyano, cyanoalkyl (e.g., acetonitrile), azido, C₁₋₆alkyl, C₃₋₈carbocyclyl, halo, —C(O)R⁵, —OC(O)R⁵, S(O)ₚR⁴, C₂₋₆alkenyl, C₂₋₆alkynyl, heterocyclyl, —OR⁷ and NR⁸R⁹, wherein R³ and R^{3′} are optionally substituted with one or more R′.
1.29 Compounds of Formula I, Formula II or any of 1.1-1.28, wherein R³ and R^{3′} are independently selected from a group consisting of hydrogen, C₁₋₆alkyl, hydroxyC₁₋₆alkyl (e.g., hydroxymethyl), alkoxy (e.g., methoxy) and haloC₁₋₆alkyl.
1.30 Compounds of Formula I, Formula II or any of 1.1-1.29, wherein R³ and R^{3′} are independently hydrogen, methyl, hydroxymethyl, methoxy, iodomethyl or fluoromethyl.
1.31 Compounds of Formula I, Formula II or any of 1.1-1.30, wherein both R³ and R^{3′} are hydroxymethyl.
1.32 Compounds of Formula I, Formula II or any of 1.1-1.30, wherein both R³ and R^{3′} are fluoromethyl.
1.33 Compounds of Formula I, Formula II or any of 1.1-1.30, wherein R³ is iodomethyl and R^{3′} is methoxy.
1.34 Compounds of Formula I, Formula II or any of 1.1-1.30, wherein one of R³ or R^{3′} is hydroxymethyl wherein said hydroxymethyl group is trans to R^{a}.
1.35 Compounds of Formula I, Formula II or any of 1.1-1.30, wherein one of R³ or R^{3′} is methyl and said methyl group is trans to R¹.
1.36 Compounds of Formula I, Formula II or any of 1.1-1.35 selected from any of the following:

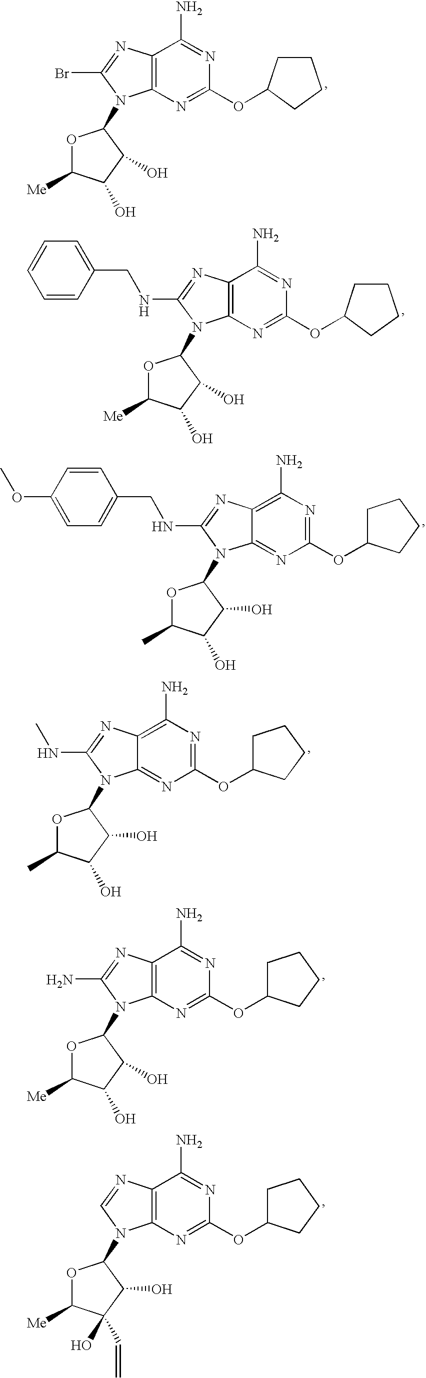

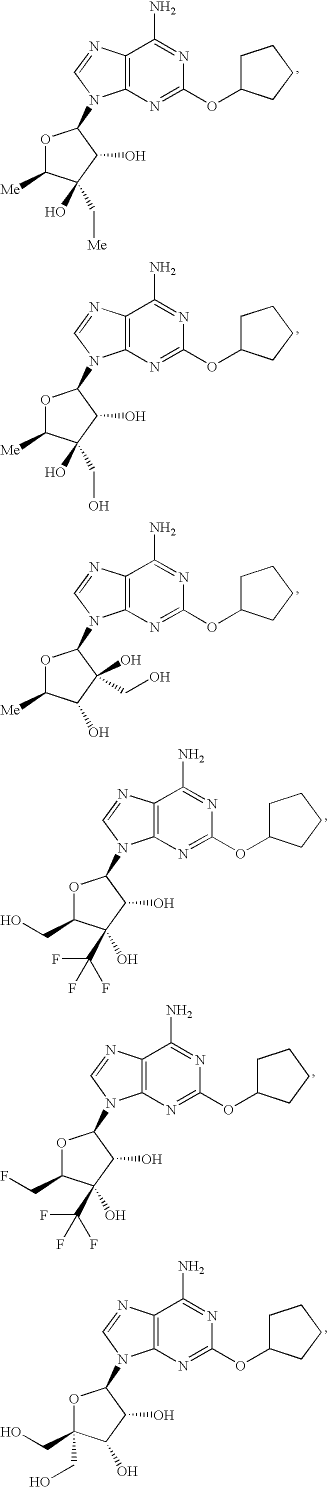

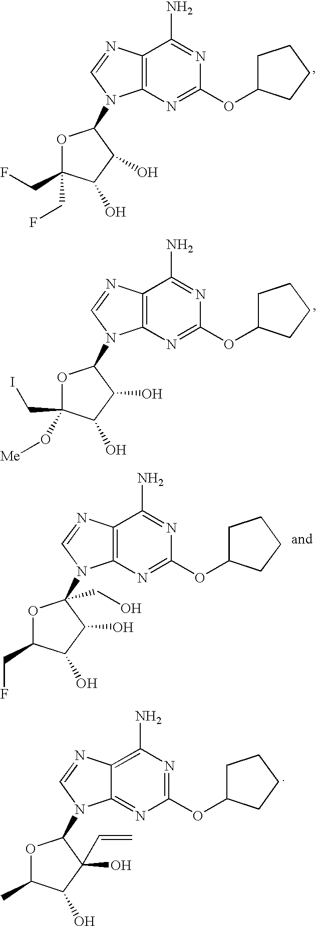

In one embodiment, therefore, the compound of formula I or Formula II is preferably

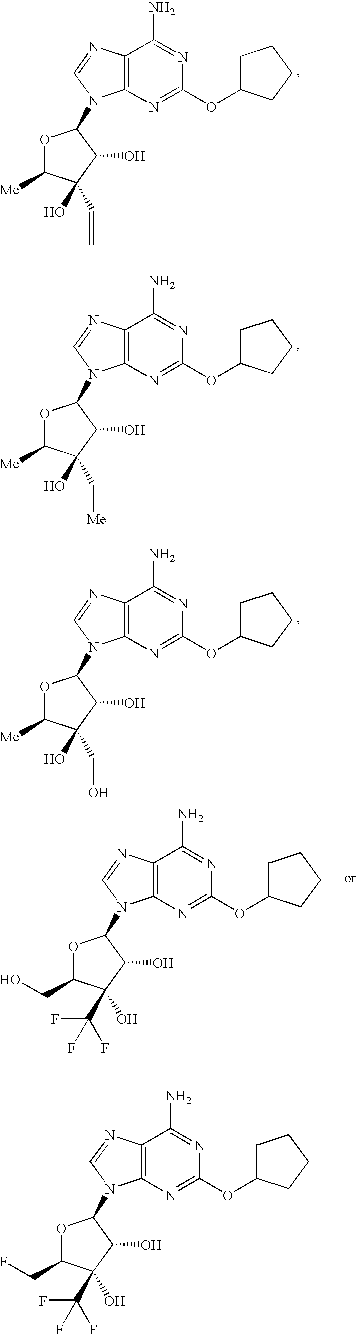

in free or salt form.

For example, in one embodiment, the compound of formula I or Formula II is

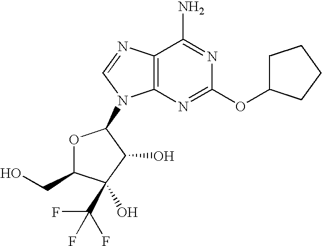

in free or salt form.

In another embodiment the compound of formula II is not (2R,3S,4R,5R)-2-(6-amino-2-(cyclopentyloxy)-9H-purin-9-yl)-5-methyl-3-vinyltetrahydrofuran-3,4-diol.

In a still further embodiment the compound of formula I is not (2R,3S,4R,5R)-2-(6-amino-2-(cyclopentyloxy)-9H-purin-9-yl)-5-methyl-3-vinyltetrahydrofuran-3,4-diol or 2-(cyclopentyloxy)-9-(3,3-dichlorotetrahydrofuran-2-yl)-9H-purin-6-amine.

In another embodiment, the compounds of formula I or formula II in free or salt form do not include compounds represented by formula IA

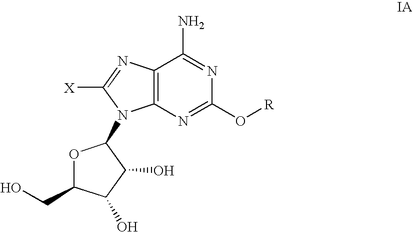

in free or salt form, wherein X and R are as defined in formula I.

In a still further embodiment, the invention provides a compound of formula I or formula II, in free or salt form, wherein

R is C₃₋₈carbocyclyl;

X is selected from hydrogen, halo or NR⁸R⁹, provided that when X is hydrogen at least one carbon is selected from C-a, C-b, C-c, and C-d is quaternary;

R¹, R^{1′}, R², R^{2′}, R³, and R^{3′} are each independently selected from hydrogen, hydroxy, C₁₋₆alkyl or C₂₋₆alkenyl, wherein R¹, R^{1′}, R², R^{2′}, R³, and R^{3′} may be optionally substituted on one or more carbon atoms by one or more R′;

R^{a} is hydrogen or C₁₋₆alkyl wherein R^{a} may be optionally substituted on one or more carbon atoms by R′;

R⁸ and R⁹ are each independently selected from hydrogen, C₁₋₆alkyl, C₂₋₆alkenyl, C₂₋₆alkynyl, —OR⁷, C₃₋₈cycloalkyl, C₃₋₈cycloalkenyl, heterocyclyl, and aryl, wherein each R⁸ or R⁹ may be optionally substituted on one or more carbon atoms by one or more R′;

R′ at each occurrence is independently halo, hydroxy, nitro, —NR⁸R⁹, azido, cyano, isocyano, C₁₋₆alkyl, C₂₋₆alkenyl, C₂₋₆alkynyl, aryl, C₃₋₈cycloalkyl, C₃₋₈cycloalkenyl, heterocyclyl, keto(═O), —OR⁷, —C(O)R⁵, —OC(O)R⁵, S(O)ₚR⁴; ═N—O—R⁶, —NHC(O)NR⁸R⁹, —N(C₁₋₆alkyl)C(O)NR⁸R⁹, —NHC(O)R⁷, —NHCO₂R⁷, —NHSO₂(R⁴), -amidino i.e. —NHC(NH)NH₂, wherein each R′ may be optionally substituted on one or more carbon by one or more R″; and

R″ at each occurrence is independently halo, azido, cyano, C₁₋₆alkyl, C₂₋₆alkenyl, C₂₋₆alkynyl, aryl, cycloalkyl, cycloalkenyl, heterocyclyl, hydroxy, —OR⁷, —C(O)R⁵, —OC(O)R⁵, S(O)ₚR⁴, —NR⁸R⁹, -amidino i.e. —NHC(NH)NH₂.

In a further embodiment, the invention provides a compound of formula I in free or salt form wherein

R is cyclopentyl;

X is selected from hydrogen, halo or NR⁸R⁹, provided that when X is hydrogen at least one carbon is selected from C-a, C-b, C-c, and C-d is quaternary;

R¹, R^{1′}, R², R^{2′}, R³, and R^{3′} are each independently selected from hydrogen, hydroxy, C₁₋₆alkyl or C₂₋₆alkenyl, wherein R¹, R^{1′}, R², R^{2′}, R³, and R^{3′} may be optionally substituted on one or more carbon atoms by one or more R′;

R^{a} is hydrogen or C₁₋₆alkyl wherein R^{a} may be optionally substituted on one or more carbon atoms by R′;

R⁸ and R⁹ are each independently selected from hydrogen, C₁₋₆alkyl, C₂₋₆alkenyl, C₂₋₆alkynyl, —OR⁷, C₃₋₈cycloalkyl, C₃₋₈cycloalkenyl, heterocyclyl, and aryl, wherein each R⁸ or R⁹ may be optionally substituted on one or more carbon atoms by one or more R′;

R′ at each occurrence is independently halo, hydroxy, nitro, —NR⁸R⁹, azido, cyano, isocyano, C₁₋₆alkyl, C₂₋₆alkenyl, C₂₋₆alkynyl, aryl, C₃₋₈cycloalkyl, C₃₋₈cycloalkenyl, heterocyclyl, keto(═O), —OR⁷, —C(O)R⁵, —OC(O)R⁵, S(O)ₚR⁴; ═N—O—R⁶, —NHC(O)NR⁸R⁹, —N(C₁₋₆alkyl)C(O)NR⁸R⁹, —NHC(O)R⁷, —NHCO₂R⁷, —NHSO₂(R⁴), -amidino i.e. —NHC(NH)NH₂, wherein each R′ may be optionally substituted on one or more carbon by one or more R″; and

R″ at each occurrence is independently halo, azido, cyano, C₁₋₆alkyl, C₂₋₆alkenyl, C₂₋₆alkynyl, aryl, cycloalkyl, cycloalkenyl, heterocyclyl, hydroxy, —OR⁷, —C(O)R⁵, —OC(O)R⁵, S(O)ₚR⁴, —NR⁸R⁹, -amidino i.e. —NHC(NH)NH₂.

In another embodiment the invention provides a compound of formula A, useful, e.g., as intermediates for the production of Compounds of Formula I or Formula II

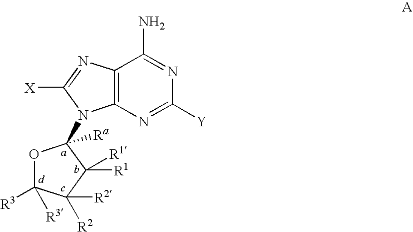

in free or salt form, wherein:

X is halo, NR⁸R⁹, azido, cyano, isocyano, hydroxy, C₁₋₆alkyl, C₂₋₆alkenyl, C₂₋₆alkynyl, aryl, C₃₋₈cycloalkyl, C₃₋₈cycloalkenyl, heterocyclyl, —OR⁷, —C(O)R⁵, —OC(O)R⁵, S(O)ₚR⁴, —NHC(O)NR⁸R⁹, —N(C₁₋₆alkyl)C(O)NR⁸R⁹, —NHC(O)R⁷, —NHCO₂R⁷, —NHSO₂(R⁴), -amidino i.e. —NHC(NH)NH₂; provided that when X is hydrogen, at least one carbon selected from C-a, C-b, C-c, and C-d is quaternary;

Y is a leaving group, e.g., halo or SO₂alkyl (e.g., SO₂Me);

p is independently at each occurrence 0, 1 or 2;

R^{a} is hydrogen, C₁₋₆alkyl, C₂₋₆alkenyl, hydroxy(C₁₋₆alkyl), and cyano, any of which may be optionally substituted on one or more carbon atom by R′;

R¹, R^{1′}, R², R^{2′}, R³, and R^{3′} are each independently selected from hydrogen, hydroxy, cyano, azido, C₁₋₆alkyl, C₃₋₈carbocyclyl, halo, —C(O)R⁵, —OC(O)R⁵, S(O)ₚR⁴, C₂₋₆alkenyl, C₂₋₆alkynyl, heterocyclyl, —OR⁷, NR⁸R⁹, wherein R¹, R^{1′}, R², R^{2′}, R³, and R^{3′} may be optionally substituted on one or more carbon atoms by one or more R′; or alternatively,

R¹ and R^{1′}, R² and R^{2′}, or R³ and R^{3′}, taken together with the carbon to which they are attached, form C═O or C═N—O—R⁶, or an optionally substituted 3, 4, 5, 6, or 7-membered ring containing 0, 1, or 2 heteroatoms selected from O, S, NH, or N(C₁₋₆alkyl); or alternatively,

R¹ and R², R² and R³, taken together with the carbons to which they are attached, form an optionally substituted 3, 4, 5, or 6-membered ring containing 0, 1, or 2 heteroatoms selected from O, S, NH, or N(C₁₋₆alkyl);

R⁴ at each occurrence is independently —NR⁸R⁹, C₁₋₆alkyl, C₂₋₆alkenyl, C₁₋₆alkoxy, C₃₋₈cycloalkyl, heterocyclyl, and aryl wherein R⁴ may be optionally substituted on one or more carbon atoms by one or more R′;

R⁵ at each occurrence is independently hydrogen, —NR⁸R⁹, —OR⁷, C₁₋₆alkyl, C₂₋₆alkenyl, C₃₋₈cycloalkyl, C₃₋₈cycloalkenyl, heterocyclyl, and aryl wherein each R⁵ may be optionally substituted on one or more carbon atoms by one or more R′;

R⁶ at each occurrence is independently hydrogen, C₁₋₆alkyl, C₂₋₆alkenyl, C₂₋₆alkynyl, C₃₋₈cycloalkyl, C₃₋₈cycloalkenyl, heterocyclyl and aryl, wherein each R⁶ may be optionally substituted on one or more carbon atoms by one or more R′;

R⁷ at each occurrence is independently hydrogen, C₁₋₆alkyl, C₂₋₆alkenyl, C₃₋₈cycloalkyl, C₃₋₈cycloalkenyl, aryl, S(O)ₚR⁴, and heterocyclyl wherein R⁷ may be optionally substituted on one or more carbon by one or more R′;

R⁸ and R⁹ are each independently selected from hydrogen, C₁₋₆alkyl, C₂₋₆alkenyl, C₂₋₆alkynyl, —OR⁷, C₃₋₈cycloalkyl, C₃₋₈cycloalkenyl, heterocyclyl, and aryl, wherein each R⁸ or R⁹ may be optionally substituted on one or more carbon atoms by one or more R′;

R′ at each occurrence is independently halo, hydroxy, nitro, —NR⁸R⁹, azido, cyano, isocyano, C₁₋₆alkyl, C₂₋₆alkenyl, C₂₋₆alkynyl, aryl, C₃₋₈cycloalkyl, C₃₋₈cycloalkenyl, heterocyclyl, keto(═O), —OR⁷, —C(O)R⁵, —OC(O)R⁵, S(O)ₚR⁴; ═N—O—R⁶, —NHC(O)NR⁸R⁹, —N(C₁₋₆alkyl)C(O)NR⁸R⁹, —NHC(O)R⁷, —NHCO₂R⁷, —NHSO₂(R⁴), -amidino i.e. —NHC(NH)NH₂, wherein each R′ may be optionally substituted on one or more carbon by one or more R″;

R″ at each occurrence is independently halo, azido, cyano, C₁₋₆alkyl, C₂₋₆alkenyl, C₂₋₆alkynyl, aryl, cycloalkyl, cycloalkenyl, heterocyclyl, hydroxy, —OR⁷, —C(O)R⁵, —OC(O)R⁵, S(O)ₚR⁴, —NR⁸R⁹, -amidino i.e. —NHC(NH)NH₂.

The invention further provides compounds of Formula A, in free or salt form, as follows:
2.1 Compounds of Formula A, wherein R is selected from a group consisting of C₁₋₈alkyl, C₂₋₈alkenyl, C₂₋₈alkynyl, C₃₋₈carbocyclyl, aryl, and heterocyclyl wherein R is optionally substituted with one or more R′.
2.2 Compounds of Formula A or 2.1, wherein R is C₃₋₈carbocyclyl.
2.3 Compounds of Formula A or 2.1 or 2.2, wherein R is cyclopentyl.
2.4 Compounds of Formula A or any of 2.1-2.3, wherein R^{a} is selected from a group consisting of hydrogen, C₁₋₆alkyl, C₂₋₆alkenyl, hydroxy(C₁₋₆alkyl), and cyano, wherein R^{a} is optionally substituted with one or more R′.
2.5 Compounds of Formula A or any of 2.1-2.4, wherein R^{a} is hydroxymethyl.
2.6 Compounds of Formula A or any of 2.1-2.4, wherein R^{a} is hydrogen
2.7 Compounds of Formula A or any of 2.1-2.6, wherein X is selected from a group consisting of hydrogen, halo, NR⁸R⁹, azido, cyano, isocyano, hydroxy, C₁₋₆alkyl, C₂₋₆alkenyl, C₂₋₆alkynyl, aryl, C₃₋₈cycloalkyl, C₃₋₈cycloalkenyl, heterocyclyl, —OR⁷, —C(O)R⁵, —OC(O)R⁵, S(O)ₚR⁴, —NHC(O)NR⁸R⁹, —N(C₁₋₆alkyl)C(O)NR⁸R⁹, —NHC(O)R⁷, —NHCO₂R⁷, —NHSO₂(R⁴), -amidino i.e. —NHC(NH)NH₂; provided that when X is hydrogen, at least one carbon selected from C-a, C-b, C-c, and C-d is quaternary.
2.8 Compounds of Formula A or any of 2.1-2.7, wherein X is selected from a group consisting of halo and NR⁸R⁹ provided that when X is hydrogen, at least one carbon selected from C-a, C-b, C-c, and C-d is quaternary.
2.9 Compounds of Formula A or any of 2.1-2.8, wherein X is bromo, amino, alkylamino (e.g., methylamino), optionally substituted phenylmethylamino (e.g., phenylmethylamino or 4-methoxyphenylmethylamino).
2.10 Compounds of Formula A or any of 2.1-2.9, wherein X is bromo.
2.11 Compounds of Formula A or any of 2.1-2.10, wherein Y is halo or SO₂alkyl.
2.12 Compounds of Formula A or any of 2.1-2.11, wherein Y is chloro, bromo, iodo or SO₂methyl.
2.13 Compounds of Formula A or any of 2.1-2.12, wherein Y is chloro.
2.14 Compounds of Formula A or any of 2.1-2.13, wherein R¹ and R^{1′} are independently selected from a group consisting of hydrogen, hydroxy, cyano, azido, C₁₋₆alkyl, C₃₋₈carbocyclyl, halo, —C(O)R⁵, —OC(O)R⁵, S(O)ₚR⁴, C₂₋₆alkenyl, C₂₋₆alkynyl, heterocyclyl, —OR⁷ and NR⁸R⁹, wherein R¹ and R^{1′} are optionally substituted with one or more R′.
2.15 Compounds of Formula A or any of 2.1-2.14, wherein R¹ and R^{1′} are independently selected from a group consisting of hydrogen, hydroxy, hydroxyalkyl or alkenyl.
2.16 Compounds of Formula A or any of 2.1-2.15, wherein one of R¹ or R^{1′} is hydrogen, hydroxy, hydroxymethyl or ethenyl.
2.17 Compounds of Formula A or any of 2.1-2.16, wherein one of R¹ or R^{1′} is hydroxy.
2.18 Compounds of Formula A or any of 2.1-2.17, wherein one of R¹ or R^{1′} is hydroxy and said hydroxy group is cis to R^{a}.
2.19 Compounds of Formula A or any of 2.1-2.18, wherein R¹ is hydroxy and said hydroxy group is cis to R^{a} and R^{1′} is hydrogen wherein said hydrogen group is trans to R^{a}.
2.20 Compounds of Formula A or any of 2.1-2.17, wherein R¹ is hydroxy wherein said hydroxy group is trans to R^{a} and R^{1′} is hydroxymethyl wherein said hydroxymethyl group is cis to R^{a}.
2.21 Compounds of Formula A or any of 2.1-2.17, wherein R¹ is hydroxy wherein said hydroxy group is trans to R^{a} and R^{1′} is ethenyl wherein said ethenyl group is cis to R^{a}.
2.22 Compounds of Formula A or any of 2.1-2.21, wherein R² and R^{2′} are independently selected from a group consisting of hydrogen, hydroxy, cyano, cyanoalkyl (e.g., acetonitrile), azido, C₁₋₆alkyl, C₃₋₈carbocyclyl, halo, —C(O)R⁵, —OC(O)R⁵, S(O)ₚR⁴, C₂₋₆alkenyl, C₂₋₆alkynyl, heterocyclyl, —OR⁷ and NR⁸R⁹, wherein R² and R^{2′} are optionally substituted with one or more R′.
2.23 Compounds of Formula A or any of 2.1-2.22, wherein R² and R^{2′} are independently selected from a group consisting of hydrogen, hydroxy, hydroxymethyl, trifluoromethyl, ethyl and ethenyl.
2.24 Compounds of Formula A or any of 2.1-2.23, wherein one of R² or R^{2′} is hydroxy.
2.25 Compounds of Formula A or any of 2.1-2.24, wherein one of R¹ or R^{1′} and another of R² or R^{2′} are cis- or trans-diol.
2.26 Compounds of Formula A or any of 2.1-2.19 or 2.22-2.25, wherein one of R¹ or R^{1′} and another of R² or R^{2′} are cis-diol and said cis-diol is cis to R¹.
2.27 Compounds of Formula A or any of 2.1-19 or 2.22-2.26, wherein R¹ and either R¹ or R^{2′} are cis-diol wherein said cis-diol is cis to R^{a} and R^{1′} is hydrogen or trifluoromethyl wherein said hydrogen or trifluoromethyl is trans to R^{a}.
2.28 Compounds of Formula A or any of 2.1-2.2.17 or 2.20-2.25 or 2.27, wherein R¹ is hydroxy wherein said hydroxy is trans to R^{a} and R^{1′} is ethyl, ethenyl or hydroxymethyl wherein said ethyl, ethenyl or hydroxymethyl is cis to R^{a}.
2.29 Compounds of Formula A or any of 2.1-2.28, wherein R³ and R^{3′} are independently selected from a group consisting of hydrogen, hydroxy, cyano, cyanoalkyl (e.g., acetonitrile), azido, C₁₋₆alkyl, C₃₋₈carbocyclyl, halo, —C(O)R⁵, —OC(O)R⁵, S(O)ₚR⁴, C₂₋₆alkenyl, C₂₋₆alkynyl, heterocyclyl, —OR⁷ and NR⁸R⁹, wherein R³ and R^{3′} are optionally substituted with one or more R′.
2.30 Compounds of Formula A or any of 2.1-2.29, wherein R³ and R^{3′} are independently selected from a group consisting of hydrogen, C₁₋₆alkyl, hydroxyC₁₋₆alkyl (e.g., hydroxymethyl), alkoxy (e.g., methoxy) and haloC₁₋₆alkyl.
2.31 Compounds of Formula A or any of 2.1-2.30, wherein R³ and R^{3′} are independently hydrogen, methyl, hydroxymethyl, methoxy, iodomethyl or fluoromethyl.
2.32 Compounds of Formula A or any of 2.1-2.31, wherein both R³ and R^{3′} are hydroxymethyl.
2.33 Compounds of Formula A or any of 2.1-2.31, wherein both R³ and R^{3′} are fluoromethyl.
2.34 Compounds of Formula A or any of 2.1-2.31, wherein R³ is iodomethyl and R^{3′} is methoxy.
2.35 Compounds of Formula A or any of 2.1-2.31, wherein one of R³ or R^{3′} is hydroxymethyl wherein said hydroxymethyl group is trans to R^{a}.
2.36 Compounds of Formula A or any of 2.1-2.31, wherein one of R³ or R^{3′} is hydroxymethyl and the other is hydrogen.
2.37 Compounds of Formula A or any of 2.1-2.31, wherein either R³ or R^{3′} is methyl and said methyl group is trans to R^{a}.
2.38 Compounds of Formula A or any of 2.1-2.31, wherein one of R³ or R^{3′} is methyl and the other is hydrogen.
   In one embodiment, therefore, the compound of formula A is

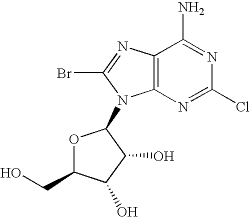

in free or salt form.

In another embodiment, the invention provides a method for making a compound of Formula 1 or Formula II, e.g., of any of Formula 1.1-1.36, comprising reacting the corresponding compound of Formula A, e.g., according to any of Formulae 2.1-2.38, with a compound of formula R—OH, wherein R is as hereinbefore described with respect to any of Formula 1 or 1.1-1.36; and recovering the compound of Formula I or Formula II.

The invention also provides a method for producing an antibacterial effect in a warm blooded animal, such as man, in need of such treatment, comprising administering to said animal an effective amount of a compound of formula I, formula II or any of 1.1-1.36 in free or pharmaceutically acceptable salt form.

The invention also provides a method for inhibition of bacterial DNA ligase in a warm-blooded animal, such as a human being, in need of such treatment comprising administering to said animal an effective amount of a compound of formula I, formula II or any of 1.1-1.36 in free or pharmaceutically acceptable salt form.

The invention also provides a method of treating a bacterial infection in a warm-blooded animal, such as a human being, in need of such treatment comprising administering to said animal an effective amount of a compound of formula I, II1.1-1.36 in free or pharmaceutically acceptable salt form.

The invention also provides a compound of formula I, formula II or any of 1.1-1.36 in free or pharmaceutically acceptable salt form, for use as a medicament.

The invention also provides the use of a compound of formula I, formula II or any of 1.1-1.36 in free or pharmaceutically acceptable salt form, in the manufacture of a medicament for use in the production of an anti-bacterial effect in a warm-blooded animal.

The invention also provides the use of a compound of formula I, formula II or any of 1.1-1.36 in free or pharmaceutically acceptable salt form, in the manufacture of a medicament for use in inhibition of bacterial DNA ligase in a warm-blooded animal, such as a human being.

The invention also provides the use of a compound of formula I, formula II or any of 1.1-1.36 in free or pharmaceutically acceptable salt form, in the manufacture of a medicament for use in the treatment of a bacterial infection in a warm-blooded animal such as a human being.

The invention also provides a compound of formula I, formula II or any of 1.1-1.36 in free or pharmaceutically acceptable salt form, for use in the production of an anti-bacterial effect in a warm-blooded animal such as a human being.

The invention also provides compound of formula I, formula II or any of 1.1-1.36 in free or pharmaceutically acceptable salt form, for use in inhibition of bacterial DNA ligase in a warm-blooded animal such as a human being.

The invention also provides a compound of formula I, formula II or any of 1.1-1.36 in free or pharmaceutically acceptable salt form, for use in the treatment of a bacterial infection in a warm-blooded animal such as a human being.

The invention also provides a pharmaceutical formulation comprising a compound of formula I, formula II or any of 1.1-1.36 in free or pharmaceutically acceptable salt form, and a pharmaceutically acceptable diluent or carrier.

The invention also provides a pharmaceutical composition comprising a compound of formula I, formula II or any of 1.1-1.36 in free or pharmaceutically acceptable salt form, in association with a pharmaceutically acceptable excipient or carrier for use in the production of an anti-bacterial effect in a warm-blooded animal, such as a human being.

### DETAILED DESCRIPTION OF THE INVENTION

The definitions set forth herein are intended to clarify terms used throughout this application unless specifically indicated otherwise. The term “herein” means the entire application.

The term “quarternary” refers to a carbon to which four non hydrogen atoms are bonded so that in the definition of “X” the phrase, “provided that when X is hydrogen, at least one carbon selected from C-a, C-b, C-c, and C-d is quaternary,” requires that when X is H then at least one of C-a, C-b, C-c, and C-d has four atoms other than hydrogen bonded to it.

The term “carbocyclyl” refers to saturated, partially saturated and unsaturated, mono, bi or polycyclic carbon rings. These may include fused or bridged bi- or polycyclic systems. Carbocyclyls may have from 3 to 12 carbon atoms in their ring structure, i.e. C₃₋₁₂carbocyclyl, and in a particular embodiment are monocyclic rings have 3 to 7 carbon atoms or bicyclic rings having 7 to 10 carbon atoms in the ring structure. Examples of suitable carbocyclyls include cyclopropyl, cyclobutyl, cyclopentyl, cyclohexyl, cycloheptyl, cyclohexenyl, cyclopentadienyl, indanyl, phenyl and naphthyl.

The term “hydrocarbon” used alone or as a suffix or prefix, refers to any structure comprising only carbon and hydrogen atoms and containing up to 12 carbon atoms.

In this specification the term alkyl, used alone or as a suffix or prefix, includes both monovalent straight and branched chain hydrocarbon radicals but references to individual alkyl radicals such as propyl are specific for the straight chain version only. An analogous convention applies to other generic terms. Unless otherwise specifically stated, the term alkyl refers to hydrocarbon radicals comprising 1 to 12 carbon atoms, in another embodiment 1 to 10 carbon atoms, and in a still further embodiment, 1 to 6 carbon atoms.

The term “alkenyl” used alone or as suffix or prefix, refers to a monovalent straight or branched chain hydrocarbon radical having at least one carbon-carbon double bond which, unless otherwise specifically stated, comprises at least 2 up to 12 carbon atoms, in another embodiment 2-10 carbon atoms and in a still further embodiment 2-6 carbon atoms.

The term “alkynyl” used alone or as suffix or prefix, refers to a monovalent straight or branched chain hydrocarbon radical having at least one carbon-carbon triple bond which, unless otherwise specifically stated, comprises at least 2 up to 12 carbon atoms, in another embodiment 2-10 carbon atoms and in a still further embodiment 2-6 carbon atoms.

In this specification, the terms alkenyl and cycloalkenyl include all positional and geometrical isomers.

The term “cycloalkyl,” used alone or as suffix or prefix, refers to a monovalent ring-containing hydrocarbon radical which, unless otherwise specifically stated, comprises at least 3 up to 12 carbon atoms, in another embodiment 3 up to 10 carbon atoms and includes monocyclic as well as bicyclic and polycyclic ring systems. When a cycloalkyl ring contains more than one ring, the rings may be fused or unfused. Fused rings generally refer to at least two rings sharing two atoms there between. Spiro rings generally refer to at least two rings sharing one atom there between. Suitable examples include C₃-C₁₀ cycloalkyl rings, e.g. cyclopropyl, cyclobutyl, cyclopentyl, cyclohexyl and cyclooctyl radicals, adamantanyl, norbornyl, decahydronapthyl, octahydro-1H-indenyl, spiro [2.2] pentanyl, and bicyclo [3.1.0] hexanyl.

The term “cycloalkenyl” used alone or as suffix or prefix, refers to a monovalent ring-containing hydrocarbon radical having at least one carbon-carbon double bond and unless otherwise specifically stated comprising at least 3 up to 12 carbon atoms, in another embodiment 3 up to 10 carbon atoms. Suitable examples include cyclopentenyl and cyclohexenyl.

The term “aryl” used alone or as suffix or prefix, refers to a hydrocarbon radical having one or more polyunsaturated carbon rings having aromatic character, (e.g., 4n+2 delocalized electrons) and comprising 5 up to 14 carbon atoms, wherein the radical is located on a carbon of the aromatic ring. Examples of suitable aryl radicals include phenyl, napthyl, and indanyl.

The term “alkoxy” used alone or as a suffix or prefix, refers to radicals of the general formula —O—R, wherein —R is selected from an optionally substituted hydrocarbon radical. Exemplary alkoxy includes methoxy, ethoxy, propoxy, isopropoxy, butoxy, t-butoxy, isobutoxy, cyclopropylmethoxy, alkyloxy, and propargyloxy.

The terms “heterocyclic radical” or “heterocyclyl” (both referred to herein as “heterocyclyl”) used alone or as a suffix or prefix, refer to a ring-containing structure or molecule having one or more multivalent heteroatoms, independently selected from N, O, and S, as a part of the ring structure and, unless otherwise specifically stated, including at least 3 and up to 14 atoms in the ring(s), or from 3-10 atoms in the ring, or from 3-6 atoms in the ring. Heterocyclyl groups may be saturated or unsaturated, containing one or more double bonds, and heterocyclyl groups may contain more than one ring. When a heterocyclyl contains more than one ring, the rings may be fused or unfused. Fused rings generally refer to at least two rings sharing two atoms therebetween. Heterocycle groups also include those having aromatic character. Examples of suitable heterocycles include, but are not limited to, indazole, pyrrolidonyl, dithiazinyl, pyrrolyl, indolyl, piperidonyl, carbazolyl, quinolizinyl, thiadiazinyl, acridinyl, azepane, azetidine, aziridine, azocinyl, benzimidazolyl, benzofuran, benzofuranyl, benzothiofuranyl, benzothiophenyl, benzoxazole, benzoxazolyl, benzthiophene, benzthiazolyl, benzotriazolyl, benzotetrazolyl, benzisoxazolyl, benzthiazole, benzisothiazolyl, benzimidazoles, benzimidazalonyl, carbazolyl, β-carbolinyl, chromanyl, chromenyl, cinnolinyl, decahydroquinolinyl, dioxanyl, dioxolanyl, furyl, dihydrofuranyl, tetrahydrothiopyranyl, furanyl, furazanyl, homopiperidinyl, imidazole, imidazolidine, imidazolidinyl, imidazolinyl, imidazolyl, indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, keto, morpholinyl, naphthyridinyl, octahydroisoquinolinyl, oxadiazolyl, oxazolidinyl, oxazolyl, oxirane, oxazolidinylperimidinyl, oxetanyl, phenanthridinyl, phenanthrolinyl, phenarsazinyl, phenazinyl, phenothiazinyl, phenoxathiinyl, phenoxazinyl, phthalazinyl, piperazinyl, piperidine, piperidinyl, pteridinyl, piperidonyl, 4-piperidonyl, purinyl, pyranyl, pyrrolidine, pyrroline, pyrrolidine, pyrrolidine-2-onyl, pyrazinyl, pyrazolidinyl, pyrazolinyl, pyrazolyl, pyridazinyl, pyridooxazole, pyridoimidazole, pyridothiazole, pyridinyl, N-oxide-pyridinyl, pyridyl, pyrimidinyl, pyrrolidinyl, pyrrolinyl, pyrrolyl, pyridine, quinazolinyl, quinolinyl, 4H-quinolizinyl, quinoxalinyl, quinuclidinyl, carbolinyl, tetrahydrofuranyl, tetrahydroisoquinolinyl, tetrahydropyranyl, thiophane, thiotetrahydroquinolinyl, thiadiazinyl, thiadiazolyl, thianthrenyl, thiazolyl, thienyl, thienothiazolyl, thienooxazolyl, thienoimidazolyl, thiophenyl, thiirane, triazinyl, triazolyl, and xanthenyl. It is further to be understood that heterocyclyl may be optionally substituted on carbon as indicated hereinbefore. Finally, if a heterocyclyl contains an —NH— moiety, the nitrogen of that moiety may be optionally substituted by a group selected from C₁₋₆alkyl, C₂₋₆alkenyl, C₂₋₆alkynyl, aryl, C₃₋₈cycloalkyl, C₃₋₈cycloalkenyl, heterocyclyl, C(O)R⁵, S(O)ₚC₁₋₆alkyl, —C(O)NR⁸R⁹, wherein the variables are as defined hereinbefore.

“Halo” includes fluorine, chlorine, bromine and iodine.

As used herein, the term “optionally substituted,” means that substitution is optional and therefore it is possible for the designated substituent to be unsubstituted. In the event a substitution is desired then such substitution means that any number of hydrogens on the designated substituent is replaced with a selection from the indicated group, provided that the normal valency of the atoms on a particular substituent is not exceeded, and that the substitution results in a stable compound. For example when a substituent is keto (i.e., ═O), then 2 hydrogens on the atom are replaced. In the case of cyclic substituents, e.g. cycloalkyl and aryl, two hydrogens may be replaced to form a second ring resulting in an overall fused or spiro ring system which may be partially or fully saturated, unsaturated or aromatic. Suitable substituents include alkylamido, e.g. acetamido, propionamido; alkyl; alkylhydroxy; alkenyl; alkenyloxy; alkynyl; alkoxy; halo; haloalkyl; hydroxy; cycloalkyl; alkylcycloalkyl; acyl; aryl; acyloxy; amino; amido; carboxy; carboxy derivatives e.g. —CONH₂, —CO₂H, —COalkyl, —COaryl, —COcycloalkyl, —COcycloalkenyl, —COheterocyclyl; substituted —NH₂; aryloxy; nitro; cyano; azido, heterocyclyl; thiol; imine; sulfonic acid; sulfate; sulfonyl; sulfinyl; sulfanyl; sulfamoyl; thioester; thioether; acid halide; anhydride; oxime i.e. ═N—OH; hydrazine; carbamate; or any other viable functional group provided that the resulting compound is stable and exhibits bacterial DNA ligase inhibitory activity. These optional substituents may themselves be optionally substituted again as long as the resulting compound is stable and exhibits a bacterial DNA ligase inhibitory effect.

When a particular substituent is indicated to be “substituted”, then that substituent can be substituted with any of the optional substituents listed above provided the resulting compound is stable and exhibits a bacterial DNA ligase inhibitory effect. Moreover, it is to be understood that combinations of substituents and/or variables are permissible only if such combinations result in stable compounds. Finally, when any variable occurs more than one time in any formula for a compound, its definition at each occurrence is independent of its definition at every other occurrence.

Where R¹ and R², R² and R³, together with the carbons to which they are attached form an optionally substituted cyclic ring containing 3-6 atoms, the cyclic ring can be a carbocyclic or heterocyclic ring. Suitable optionally substituted carbocyclic and heterocyclic rings include, cyclic ethers e.g. epoxide, oxetanyl, dioxanyl, e.g. 2,2-dimethyl-1,3-dioxanyl; cycloalkyl rings e.g. cyclopropyl, cyclobutyl, cyclopentyl and cyclohexyl, cyclohexanonyl rings; heterocyclyl rings e.g. azetidinyl, oxazolidonyl ring, oxathiolanyl ring, oxazinonyl ring, pyranonyl ring, piperidinonyl, tetrahydrothiophenyl ring, pyrrolidinyl ring, dioxolanyl ring, dioxanonyl ring, triazolyl ring, tetrazolyl ring, morpholinyl ring, 1,3,2-dioxathiolane-2,2-dioxidyl ring and piperidinyl ring.

The terms “cis” and “trans” are well known in the art and generally refer to the relative orientation of two substituents on a double bond or a cyclic compound. As applied to the compounds of the present invention, they refer to the orientation of R^{a}, R¹, R^{1′}, R², R^{2′}, R³ and R^{3′} relative to each other on the tetrahydrofuran ring. Therefore, “cis” refers to two substituents that are on the same side of the plane of the ring (e.g., both above or both below the plane of the ring) while “trans” refers to two substituents that are on different side of the plane of the ring (e.g., one above and one below the plane of the ring). For example, since R¹ and R^{1′} have not been designated a specific orientation, the phrase “wherein one of R¹ or R^{1′} is hydroxy and said hydroxy group is cis to R^{a}” refers to compounds wherein the hydroxy group on the b-carbon is on the same side of the plane of the ring as R^{a} that is on the a-carbon. Similarly, “wherein one of R¹ or R^{1′} and another R² or R^{2′} are cis- or trans-diol” refers to compounds wherein the hydroxy group on the b-carbon is on the same side of the plane of the ring as the hydroxy group on the c-carbon (i.e., cis-diol) or compounds wherein the hydroxy group on the b-carbon is on a different side of the plane of the ring as the hydroxy group on the c-carbon (i.e., trans-diol). Therefore, cis- or trans-diol may be illustrated below as examples only:

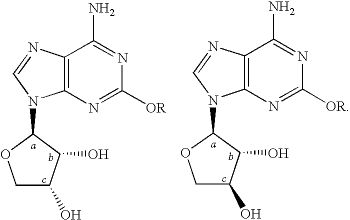

Furthermore, the phrase “wherein one of R³ or R^{3′} is methyl and said methyl group is trans to R^{a}” refers to compounds wherein the methyl group on the d-carbon is on a different side of the plane of the ring as R^{a} that is on the a-carbon as illustrated below by way of examples only:

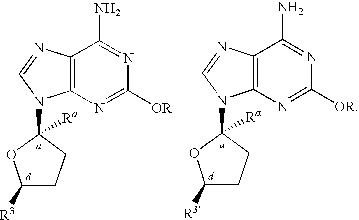

wherein R³ or R^{3′} is methyl

“Pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “base” herein refers to organic or inorganic bases such as hydrides (e.g. sodium or potassium hydride); alkoxides, (e.g. sodium, potassium or lithium t-butoxide); or carbonate, bicarbonate, phosphate or hydroxide of an alkali or alkaline earth metal (e.g. sodium, magnesium, calcium, potassium, cesium or barium). Examples of organic bases include neutral or anionic amine bases.

Compounds of the foregoing formulas I and Formula II may form stable acid or basic salts, and in such cases administration of a compound as a salt may be appropriate, and pharmaceutically acceptable salts may be made by conventional methods well-known in the art.

Suitable pharmaceutically-acceptable salts include acid addition salts such as methanesulfonate, trifluoroacetate, tosylate, α-glycerophosphate fumarate, hydrochloride, citrate, maleate, tartrate and hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl D-glucamine and amino acids such as lysine. There may be more than one cation or anion depending on the number of charged functions and the valency of the cations or anions.

However, to facilitate isolation of the salt during preparation, salts which are less soluble in the chosen solvent may be preferred whether pharmaceutically-acceptable or not.

Within the present invention it is to be understood that a compound of formula I or a salt thereof may exhibit the phenomenon of tautomerism and that the formula drawings within this specification can represent only one of the possible tautomeric forms. It is to be understood that the invention encompasses all tautomeric forms that inhibit bacterial DNA ligase and is not to be limited merely to any one tautomeric form utilized within the formula drawings.

It will be appreciated by those skilled in the art that in addition to the asymmetric carbon atoms specifically indicated in formula I, the compounds of the of the invention may contain additional asymmetrically substituted carbon and/or sulphur atoms, and accordingly may exist in, and be isolated in, optically-active and racemic forms. Some compounds may exhibit polymorphism. It is to be understood that the present invention encompasses any racemic, optically-active, polymorphic or stereoisomeric form, or mixtures thereof, which possesses properties useful in the inhibition of bacterial DNA ligase, it being well known in the art how to prepare optically-active forms (for example, by resolution of the racemic form by recrystallization techniques, by synthesis from optically-active starting materials, by chiral synthesis, by enzymatic resolution, by biotransformation, or by chromatographic separation using a chiral stationary phase) and how to determine efficacy for the inhibition of bacterial DNA ligase by the standard tests described hereinafter.

When an optically active form of a compound of the invention is required, it may be obtained as specifically exemplified above or by carrying out one of the above procedures for racemic compounds but using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

It is also to be understood that compounds of formula I and salts thereof can exist in solvated as well as unsolvated forms such as, for example, hydrated forms. It is to be understood that the invention encompasses all such solvated forms that inhibit bacterial DNA ligase.

The removal of any protecting groups and the formation of pharmaceutically acceptable salts are within the skill of an ordinary organic chemist using standard techniques.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients well known in the art. Thus, compositions intended for oral use may contain, for example, one or more coloring, sweetening, flavoring and/or preservative agents.

Suitable pharmaceutically acceptable excipients for a tablet formulation include, for example, inert diluents such as lactose, sodium carbonate, calcium phosphate or calcium carbonate; granulating and disintegrating agents such as corn starch or algenic acid; binding agents such as starch; lubricating agents such as magnesium stearate, stearic acid or talc; preservative agents such as ethyl or propyl p-hydroxybenzoate; and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form or in the form of nano or micronized particles together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives such as ethyl or propyl p-hydroxybenzoate; anti-oxidants such as ascorbic acid); colouring agents; flavouring agents; and/or sweetening agents such as sucrose, saccharine or aspartame.

Oily suspensions may be formulated by suspending the active ingredient in a vegetable oil such as arachis oil, olive oil, sesame oil or coconut oil or in a mineral oil such as liquid paraffin. The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

In addition to the compounds of the present invention, the pharmaceutical composition of this invention may also contain or be co-administered (simultaneously, sequentially or separately) with one or more known drugs selected from other clinically useful antibacterial agents (for example, macrolides, quinolones, β-lactams or aminoglycosides) and/or other anti-infective agents (for example, an antifungal triazole or amphotericin). These may include carbapenems, for example meropenem or imipenem, to broaden the therapeutic effectiveness. Compounds of this invention may also contain or be co-administered with bactericidal/permeability-increasing protein (BPI) products or efflux pump inhibitors to improve activity against gram negative bacteria and bacteria resistant to antimicrobial agents.

As stated above the size of the dose required for the therapeutic or prophylactic treatment of a particular disease state will necessarily be varied depending on the host treated, the route of administration and the severity of the illness being treated. Preferably a daily dose in the range of 1-50 mg/kg is employed. Accordingly, the optimum dosage may be determined by the practitioner who is treating any particular patient.

In addition to its use in therapeutic medicine, compounds of formula I and their pharmaceutically acceptable salts are also useful as pharmacological tools in the development and standardization of in vitro and in vivo test systems for the evaluation of the effects of inhibitors of DNA ligase in laboratory animals such as cats, dogs, rabbits, monkeys, rats and mice, as part of the search for new therapeutic agents.

In any of the above-mentioned pharmaceutical composition, method, use, medicament, and manufacturing features of the instant invention, any of the alternate embodiments of the compounds of the invention described herein also apply.

### Enzyme Potency Testing Methods

Compounds were tested for inhibition of DNA ligase using a Fluorescence Resonance Energy Transfer (FRET) detection assay as previously described (Chen et al. 2002. Analytical Biochemistry 309: 232-240; Benson et al. 2004. Analytical Biochemistry 324:298-300). Assays were performed in 384-well polystyrene flat-bottom black plates in 30 μl reactions containing 3 μl compound dissolved in dimethylsulfoxide, 20 μl 1.5× Enzyme Working Solution (25% glycerol, 45 mM potassium chloride, 45 mM ammonium sulfate, 15 mM dithiothreitol, 1.5 mM ethylenediaminetetraacetic acid (EDTA), 0.003% Brij 35, 75 mM MOPS pH 7.5, 150 nM bovine serum albumin, 1.5 μM NAD⁺, 60 nM DNA substrate, 0.375 nM enzyme in water) and 7 μl 70 mM magnesium chlorine solution (96 mM magnesium chloride, 20% glycerol in water) to initiate the reaction. The DNA substrate is similar to that described in Benson et al. (2004. Analytical Biochemistry 324:298-300). The assay reactions were incubated at room temperature for approximately 20 minutes before being terminated by the addition of 30 μl Quench reagent (8 M Urea, 1 M Trizma base, 20 mM EDTA in water). Plates were read in a Tecan Ultra plate reader at two separate wavelengths—Read 1: excitation 485, emission 535, Read 2: excitation 485, emission 595. Data is initially expressed as a ratio of the 595/535 emission values and percent inhibition values were calculated using 0.2% dimethylsulfoxide (no compound) as the 0% inhibition and EDTA-containing (50 mM) reactions as 100% inhibition controls. Compound potency was based on IC₅₀ measurements determined from reactions performed in the presence of ten different compound concentrations.

The compounds described have a measured IC₅₀ in this assay against at least one isozyme (S. pneumoniae, S. aureus, H. influenzae, E. coli, or M. pneumoniae) of <400 μM or the compounds inhibited the ligation reaction by >20% at the limit of their solubility in the assay medium. Solubility is determined under assay conditions using a nephelometer to detect a change in turbidity as the concentration of compound increases. The limit of solubility is defined as the maximum concentration before a detectable increase in turbidity is measured.

Representative bacterial DNA ligase inhibition by the compounds of the instant invention is indicated below.

DNA Ligase Inhibitory Activity

| Example Number | IC₅₀ (μM) against S. pneumoniae LigA |
|---|---|
| 2 | 10 |
| 13 | 1 |

2-(cyclopentyloxy)-9-(5-deoxy-2-C-vinyl-b-D-arabinofuranosyl)-9H-purin-6-amine only inhibited the ligation reaction by approximately 17% at the limit of its solubility in the assay medium while 2-(cyclopentyloxy)-9-(3,3-dichlorotetrahydrofuran-2-yl)-9H-purin-6-amine only inhibited the ligation reaction by approximately 12% at the limit of its solubility in the assay medium.

### Bacterial Susceptibility Testing Methods

Compounds were tested for antimicrobial activity by susceptibility testing using microbroth dilution methods recommended by NCCLS. Compounds were dissolved in dimethylsulfoxide and tested in 10 doubling dilutions in the susceptibility assays such that the final dimethylsulfoxide concentration in the assay was 2% (v/v). The organisms used in the assay were grown overnight on appropriate agar media and then suspended in the NCCLS-recommended liquid susceptibility-testing media. The turbidity of each suspension was adjusted to be equal to a 0.5 McFarland standard, a further 1-in-10 dilution was made into the same liquid medium to prepare the final organism suspension, and 100 μL of this suspension was added to each well of a microtiter plate containing compound dissolved in 2 μL of dimethylsulfoxide. Plates were incubated under appropriate conditions of atmosphere and temperature and for times according to NCCLS standard methods prior to being read. The Minimum Inhibitory Concentration (MIC) was determined as the lowest drug concentration able to reduce growth by 80% or more.

Representative antibacterial activity for the compounds of the instant invention is indicated below.

Antibacterial Activity

| Example Number | MIC (μg/ml) against S. pneumoniae |
|---|---|
| 12 | 16 |

### Process

If not commercially available, the necessary starting materials for the procedures such as those described herein may be made by procedures which are selected from standard organic chemical techniques, techniques which are analogous to the synthesis of known, structurally similar compounds, or techniques which are analogous to the described procedure or the procedures described in the Examples.

It is noted that many of the starting materials for synthetic methods as described herein are commercially available and/or widely reported in the scientific literature, or could be made from commercially available compounds using adaptations of processes reported in the scientific literature. The reader is further referred to Advanced Organic Chemistry, 4^{th} Edition, by Jerry March, published by John Wiley & Sons 1992, for general guidance on reaction conditions and reagents.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, published by John Wiley and Sons, 1991).

Examples of suitable protecting groups for a hydroxy group are, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric, phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid, for example boron tris(trifluoroacetate). A suitable alternative protecting group for a primary amino group is, for example, a phthaloyl group, which may be removed by treatment with an alkylamine, for example dimethylaminopropylamine or 2-hydroxyethylamine, or with hydrazine. Another suitable protecting group for an amine is, for example, a cyclic ether such as tetrahydrofuran, which may be removed by treatment with a suitable acid such as trifluoroacetic acid.

The protecting groups may be removed at any convenient stage in the synthesis using conventional techniques well known in the chemical art, or they may be removed during a later reaction step or work-up.

The skilled organic chemist will be able to use and adapt the information contained and referenced within the above references, and accompanying Examples therein and also the Examples herein, to obtain necessary starting materials and products.

Another aspect of the present invention provides a process for preparing a compound of formula I or a pharmaceutically acceptable salt thereof which process (wherein R, R^{a}, R¹, R^{1′}, R², R^{2′}, R³ and R^{3′} are, unless otherwise specified, as defined in formula I) comprises:

a) reacting a purine base of formula (1):

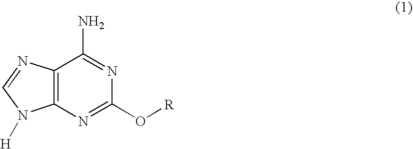

or a suitably protected derivative thereof; with an electrophile of formula (2) wherein L is a suitable leaving group such as acetate, methoxy, benzoyl, or chloro; or

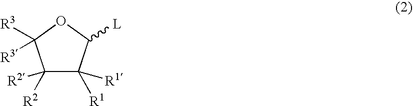

reacting a purine base of formula (3):

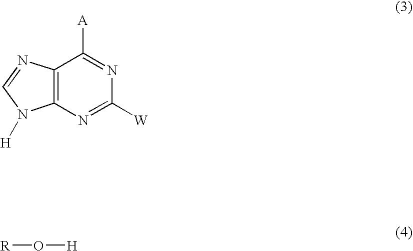

wherein A is Cl, NH₂, or a suitably protected amino group and W is halo, with an electrophile of formula (2) followed by reaction with a compound of formula (4), and if A is Cl, a subsequent reaction with the appropriate amine, such as ammonia;
and thereafter if necessary:
converting a compound of formula I into another compound of formula I; removing any protecting groups;
and optionally forming a pharmaceutically acceptable salt.

Specific reaction conditions for the above reactions are as follows:

Purine bases of formula (1) and electrophiles of formula (2) may be coupled together using standard coupling conditions known in the art. These include, but are not limited to glycosylation conditions such as those described in Vorbrueggen, H. and Bennua, B. Chem. Ber., 1981, 114, 1279-1286, and Dudycz, L. V. and Wright, G. E. Nucleosides and Nucleotides, 1984, 3, 33-44. Other coupling methods include but are not limited to nucleophilic substitution reactions catalyzed by, for example bases, Lewis acids or palladium, and substitution using reagents such as triphenylphosphine and diethylazodicarboxylate. Alternative methods of synthesizing compounds of formula I, for example starting from the appropriately substituted pyrimidine or imidazole, can be utilized as described in Joule, J. A. and Mills, K., Heterocyclic Chemistry, Fourth Edition, published by Blackwell Publishing, 2000, for analogous compounds.

A compound of formula (1) can be prepared by functionalization of a substituted purine compound which is commercially available or is a known compound or is prepared by processes known in the art, for example by processes such as those shown in Scheme 1.

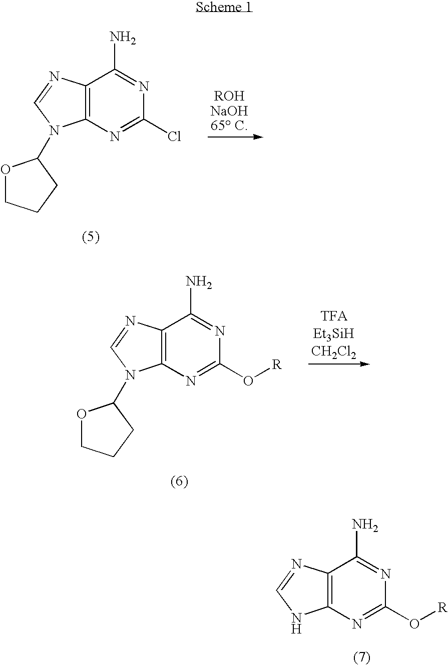

Displacement of halo by the appropriate alcohol can be done either neat or in a suitable solvent such as dioxane, tetrahydrofuran, DCM, DMF, or N-methylpyrrolidinone in temperatures ranging from 20-200° C. Bases such as sodium hydroxide, potassium carbonate, n-butyl lithium, potassium tert-butoxide, or sodium hydride can be used as necessary according to one skilled in the art. If necessary, a suitable protecting group, for example benzoyl, can be installed prior to deprotection of the tetrahydrofuran.

Compounds of formula (2) are prepared by processes known in the art using procedures found in the literature such as those modifying an appropriately protected ribose derivative. The reader is referred to Preparative Carbohydrate Chemistry, edited by S. Hanessian, published by Marcel Dekker, 1997 for general guidance on transformations and reaction conditions. For example, one method to synthesize compound (11) is shown in Scheme 2. The reaction of compound (8) or other suitably protected ribose derivative with a displaceable group can be carried out by a number of fluorinating reagents such as tetrabutylammonium fluoride, (diethylamino)sulfur trifluoride (DAST), potassium fluoride, or Amberlyst A-26 (F-40 nm) to give compound (9). Following deprotection and reprotection, compound (11) is obtained and can be coupled with a compound of formula (1).

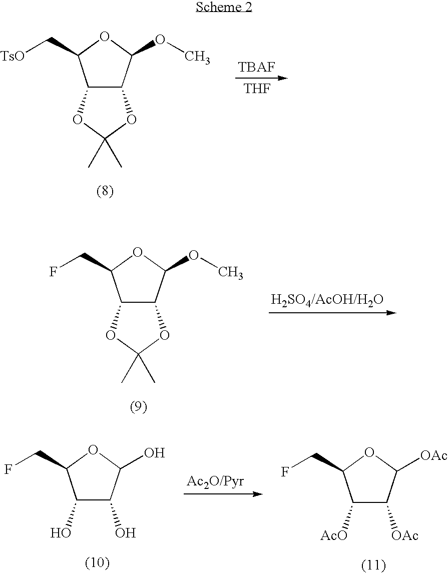

Alternatively, compounds of formula I can be prepared by converting a particular compound of formula I to a different compound of formula I using the appropriate protecting groups, reactions, and deprotections using methods known to one skilled in the art. One non-limiting example of how the 5′-position of the ribose can be modified is shown in Scheme 3, and one non-limiting example of how the 2′- and 3′-positions of the ribose can be modified is shown in Scheme 4. Appropriate chemistry can be applied to modify the 5′ and 2′ and 3′-positions of the ribose, in each case using the appropriate combination of protecting groups. Further manipulations can be made using techniques known to one skilled in the art.

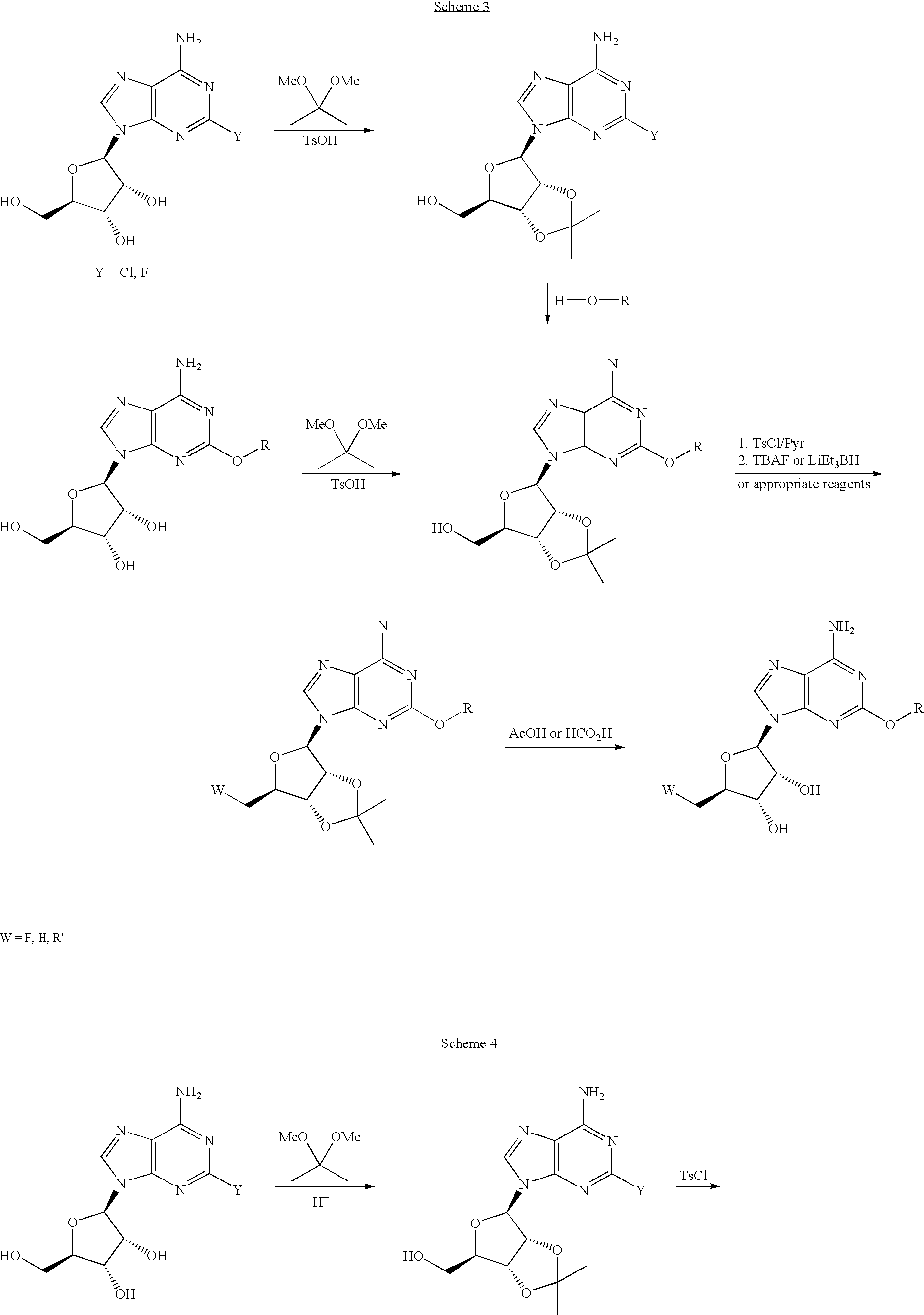

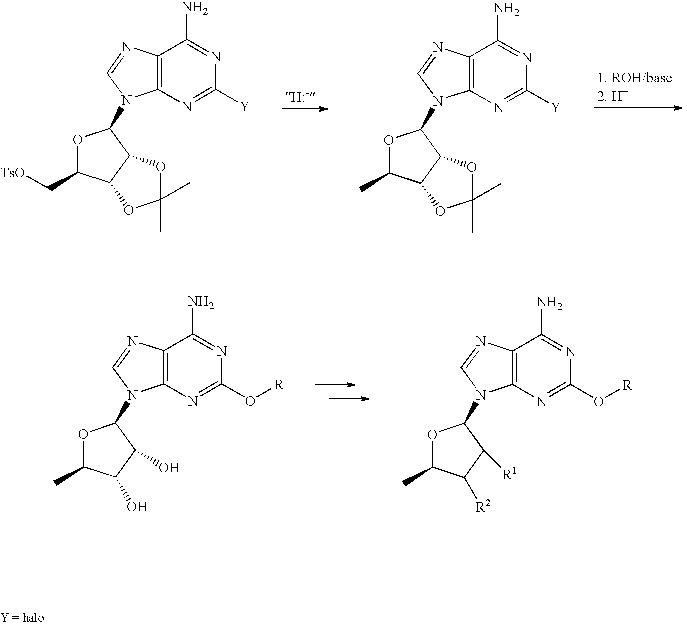

The alcohols used in the displacement reaction on the 2-haloadenosine may be commercially available. Those that aren't can be synthesized by methods well known to those of skill in the art. One non-limiting example is shown in Scheme 5.

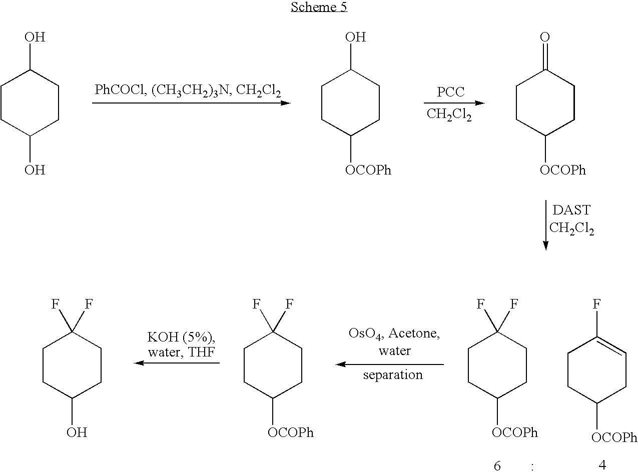

Attachment of X to complete the preparation of a compound of formula I can occur as outlined in Scheme 6.

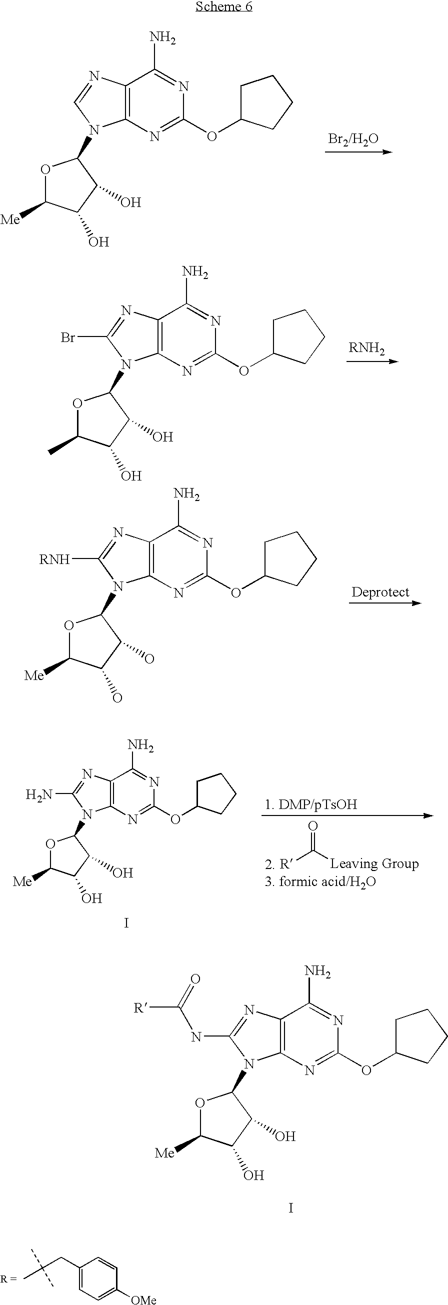

Compounds of formula I can be prepared in which at least one carbon is made quaternary by using procedures such as those shown in Schemes 7-10.

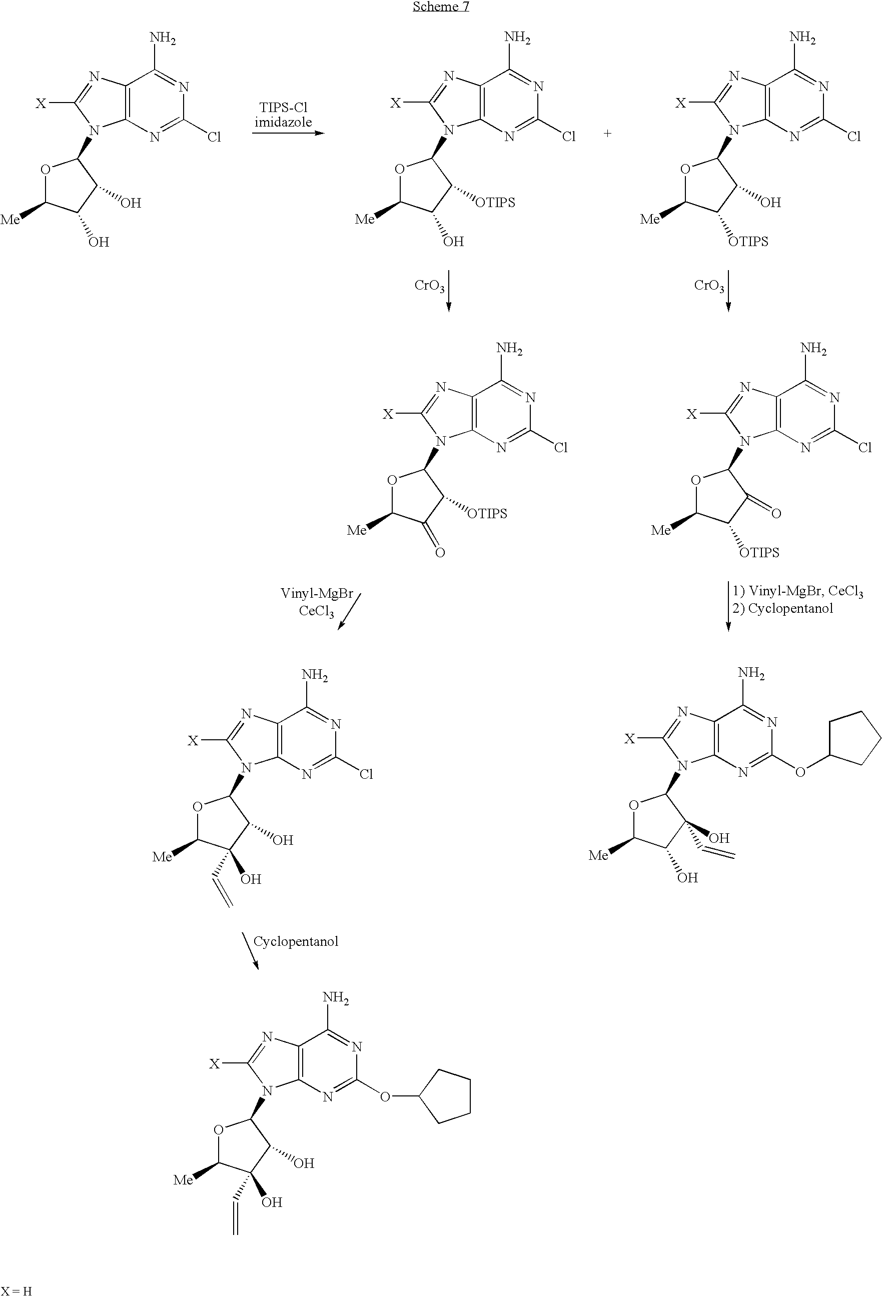

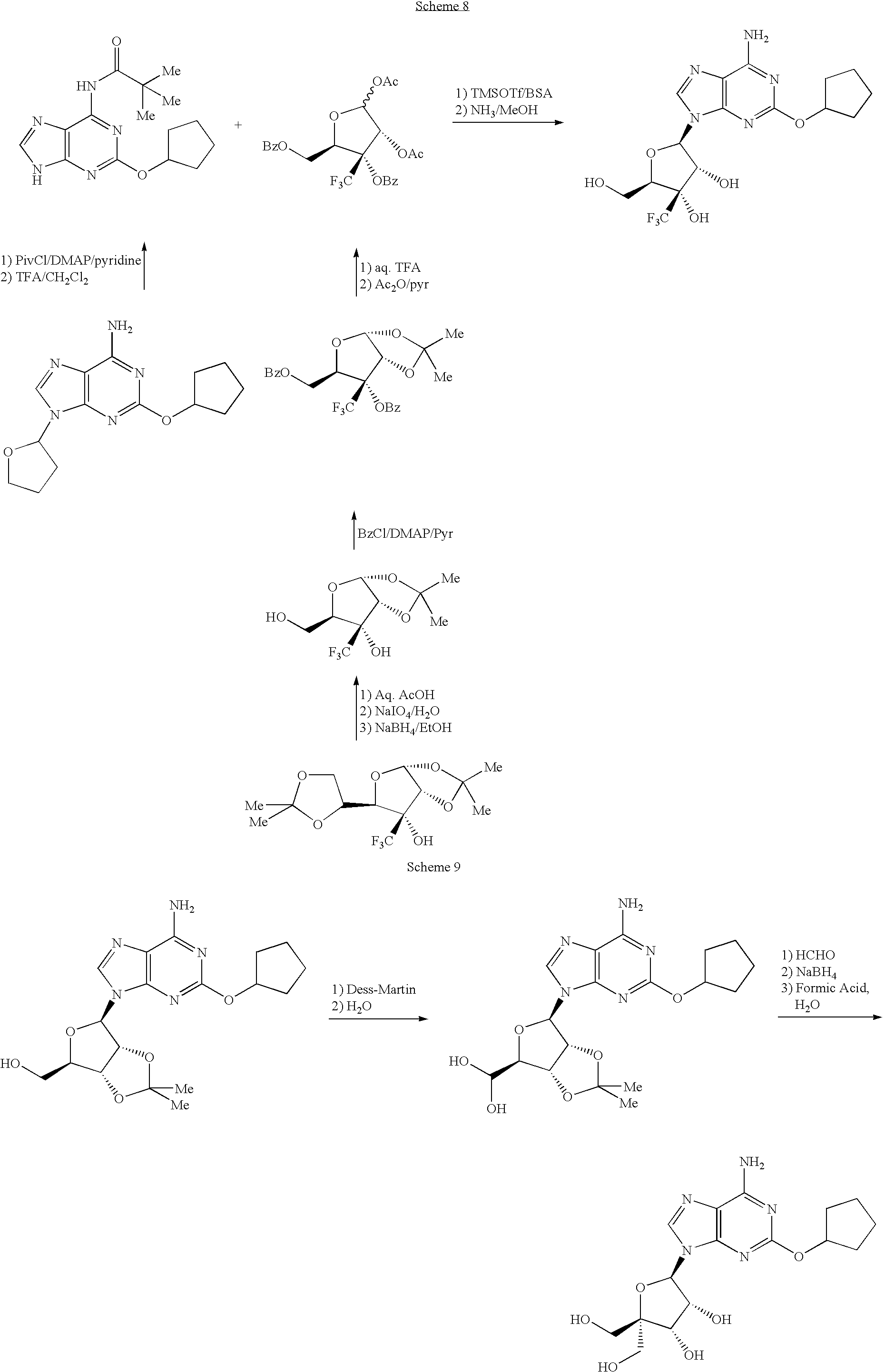

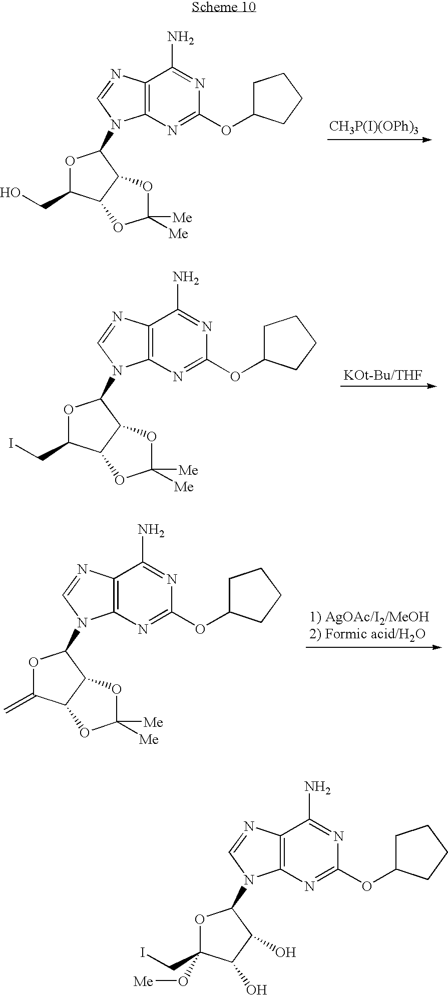

Compounds of formula A may be prepared by first treating a 2-chloro-9H-purin-6-amine with a tetrahydrofuran acetate derivative (e.g., (3R,4R,5R)-5-(hydroxymethyl)-tetrahydrofuran-2,3,4-triyl triacetate) and SnCl₄. Then, treat the resulting product (e.g., (2R,3R,4S,5R)-2-(6-amino-2-chloro-9H-purin-9-yl)-5-(hydroxymethyl)tetrahydrofuran-3,4-diol)) with a halogen (e.g., bromine) to afford for example (2R,3R,4S,5R)-2-(6-amino-8-bromo-2-chloro-9H-purin-9-yl)-5-(hydroxymethyl)tetrahydrofuran-3,4-diol.

### EXAMPLES

The invention is now illustrated but not limited by the following Examples in which unless otherwise stated:

(i) evaporations were carried out by rotary evaporation in vacuo and work-up procedures were carried out after removal of residual solids by filtration;
(ii) temperatures are quoted as ° C.; operations were carried out at room temperature, that is typically in the range 18-26° C. and without exclusion of air unless otherwise stated, or unless the skilled person would otherwise work under an inert atmosphere;
(iii) column chromatography (by the flash procedure) was used to purify compounds and was performed on Merck Kieselgel silica (Art. 9385) unless otherwise stated; Jones Flashmaster and Biotage refer to automated normal phase chromatography instruments using silica cartridges for the stationary phase; the instruments were used according to the manufacturers instructions;
(iv) in general, the course of reactions was followed by TLC, HPLC, or LC/MS and reaction times are given for illustration only; yields are given for illustration only and are not necessarily the maximum attainable;
(v) the structure of the end-products of the invention were generally confirmed by NMR and mass spectral techniques. Proton magnetic resonance spectra were generally determined in DMSO-d₆ unless otherwise stated, using a Bruker DRX-300 spectrometer operating at a field strength of 300 MHz. In cases where the NMR spectrum is complex, only diagnostic signals are reported. Chemical shifts are reported in parts per million downfield from tetramethysilane as an internal standard (δ scale) and peak multiplicities are shown thus: s, singlet; d, doublet; dd, doublet of doublets; dt, doublet of triplets; dm, doublet of multiplets; t, triplet, m, multiplet; br, broad. Fast-atom bombardment (FAB) mass spectral data were generally obtained using a Platform spectrometer (supplied by Micromass) run in electrospray and, where appropriate, either positive ion data or negative ion data were collected or using Agilent 1100 series LC/MSD equipped with Sedex 75ELSD, and where appropriate, either positive ion data or negative ion data were collected. The lowest mass major ion is reported for molecules where isotope splitting results in multiple mass spectral peaks (for example when chlorine is present). Reverse Phase HPLC was carried out using YMC Pack ODS-AQ (100×20 mmID, S-5μ particle size, 12 nm pore size) on Agilent instruments;
(vi) each intermediate was purified to the standard required for the subsequent stage and was characterized in sufficient detail to confirm that the assigned structure was correct; purity was assessed by HPLC, TLC, or NMR and identity was determined by infra-red spectroscopy (IR), mass spectroscopy or NMR spectroscopy as appropriate;
(vii) the following abbreviations may be used:
TLC is thin layer chromatography; HPLC is high pressure liquid chromatography; MPLC is medium pressure liquid chromatography; NMR is nuclear magnetic resonance spectroscopy; DMSO is dimethylsulfoxide; CDCl₃ is deuterated chloroform; MeOD is deuterated methanol, i.e. D₃COD; MS is mass spectroscopy; ESP (or ES) is electrospray; EI is electron impact; APCI is atmospheric pressure chemical ionization; THF is tetrahydrofuran; DCM is dichloromethane; MeOH is methanol; DMF is dimethylformamide; EtOAc is ethyl acetate; LC/MS is liquid chromatography/mass spectrometry; h is hour(s); min is minute(s); d is day(s); TFA is trifluoroacetic acid; v/v is ratio of volume/volume; Boc denotes t-butoxycarbonyl; Cbz denotes benzyloxycarbonyl; Bz denotes benzoyl; atm denotes atmospheric pressure; rt denotes room temperature; mg denotes milligram; g denotes gram; □L denotes microliter; mL denotes milliliter; L denotes liter; □M denotes micromolar; mM denotes millimolar; M denotes molar; N denotes normal; nm denotes nanometer;
(viii) microwave reactor refers to a Smith Microwave Synthesizer, equipment that uses microwave energy to heat organic reactions in a short period of time; it was used according to the manufacturers instructions and was obtained from Personal Chemistry Uppsala AB; and
(ix) Kugelrohr distillation refers to a piece of equipment that distills liquids and heats sensitive compounds using air-bath oven temperature; it was used according to the manufacturers instruction and was obtained from Büchi, Switzerland or Aldrich, Milwaukee, USA.

### Example 1

### 8-bromo-2-(cyclopentyloxy)-9-(5-deoxy-D-D-ribofuranosyl)-9H-purin-6-amine

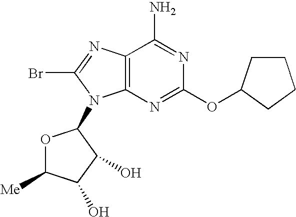

2-(Cyclopentyloxy)-9-(5-deoxy-β-D-ribofuranosyl)-9H-purin-6-amine (235 mg, 0.7 mmol) was dissolved in sodium acetate buffer (5 ml) (pH 4, 1M) at 50° C. The solution was cooled to rt, and bromine (43 mL, 0.84 mmol) was added. The reaction was stirred at rt overnight. Excess bromine was destroyed by addition of 10% sodium bisulfite (1 ml). The pH was adjusted to pH 7 with 5N sodium hydroxide. Residue was purified by RP-HPLC giving 130 mg of a white solid corresponding to the desired product.

MS (ESP): 414.22 (MH⁺) for C₁₅H₂₀BrN₅O₄

¹H NMR (300 MHz, DMSO-d₆) 8 ppm 1.26 (d, 3H); 1.57-1.89 (series of m, 8H); 3.89-3.91 (m, 1H); 4.06-4.20 (m, 1H); 5.06-5.22 (series of m, 3H); 5.42 (d, 1H); 5.67 (d, 1H); 7.37 (br s

### Example 2

### N⁸-benzyl-2-(cyclopentyloxy-9-(5-deoxy-O-D-ribofuranosyl)-9H-purine-6,8-diamine

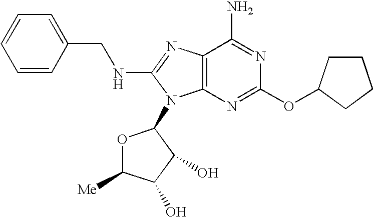

8-bromo-2-(cyclopentyloxy)-9-(5-deoxy-β-D-ribofuranosyl)-9H-purin-6-amine (0.2 g, 0.48 mmol) was dissolved in methanol (3 ml), and benzylamine (0.52 ml, 4.8 mmol) was added. The reaction was heated in a microwave reactor at 100° C. for 3 h. The volatiles were removed in vacuo; diethyl ether was added to the residue and concentrated in vacuo (repeat 5×). Purification by RP-HPLC resulted in the desired product as a white solid (5.5 mg).

MS (ESP): 441.31 (MH⁺) for C₂₂H₂₈N₆O₄

¹H NMR (300 MHz, DMSO-d₆) δ ppm 1.23 (d, 3H); 1.55-1.90 (series of m, 8H); 3.80 (m, 1H); 4.09 (m, 1H); 4.52 (d, 2H); 4.98 (d, 1H); 4.99 (m, 1H); 5.15 (m, 1H); 5.26 (d, 1H); 5.63 (m, 1H); 6.43 (br s, 2H); 7.15 (t, 1H); 7.19-7.37 (m, 5H).

Using a procedure analogous for that described for N⁸-benzyl-2-(cyclopentyloxy)-9-(5-deoxy-β-D-ribofuranosyl)-9H-purine-6,8-diamine, the following compounds were synthesized using the appropriate commercially available amine:

| EX | IUPAC name | MH+ | 1H NMR (DMSO-d6) |
|---|---|---|---|
| 3 | 2-(cyclopentyloxy)-9-(5- | 471.35 | 1.22 (d, 3 H) 1.68 (mm, 6 H) 1.89 (m, |
| | deoxy-β-D-ribofuranosyl)-N8- | | 2 H) 3.71 (s, 3 H) 3.78 (m, 1 H) 4.09 |
| | (4-methoxybenzyl)-9H-purine- | | (m, 1 H) 4.43 (d, 2 H) 4.98 (m, 2 H) |
| | 6,8-diamine | | 5.14 (m, 1 H) 5.25 (m, 1 H) 5.61 (d, 1 |
| | | | H) 6.43 (s, 2 H) 6.86 (d, 2 H) 7.05 (m, |
| | | | 1 H) 7.30 (d, 2 H) |
| 4 | 2-(cyclopentyloxy)-9-(5- | 365.3 | 1.23 (d, 3 H) 1.57 (mm, 2 H) 1.68 (m, |
| | deoxy-β-D-ribofuranosyl)-N8- | | 4 H) 1.78-1.93 (m, 2 H) 2.82 (d, 3 H) |
| | methyl-9H-purine-6,8-diamine | | 3.78 (m, 1 H) 4.09 (m, 1 H) 4.96 (d, 2 |
| | | | H) 5.14 (m, 1 H) 5.25 (d, 1 H) 5.50 (m, |
| | | | 1 H) 6.45 (s, 2 H) 6.55 (m, 1 H) |

### Example 5

### 2-(cyclopentyloxy)-9-(5-deoxy-O-D-ribofuranosyl)-9H-purine-6,8-diamine

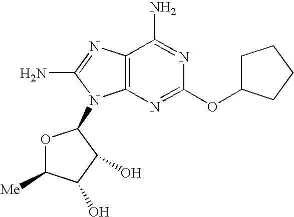

2-(Cyclopentyloxy)-9-(5-deoxy-D-ribofuranosyl)-N⁸-(4-methoxybenzyl)-9H-purine-6,8-diamine (0.07 g, 0.148 mmol), was suspended in acetonitrile (5 ml). To the mixture was added ammonium cerium (IV) nitrate (0.16 g, 0.3 mmol) and water (1 ml). The mixture was stirred for 18 h at rt. When LC/MS indicated that the reaction was complete, the solvents were removed in vacuo and purified by RP-HPLC. An off-white solid (5.6 mg, 31%) was obtained.

MS (ESP): 351.29 (MH⁺) for C₁₅H₂₂N₆O₄

¹H NMR (300 MHz, DMSO-d₆) 5 ppm 1.23 (d, 3H) 1.57 (m, 5H) 1.89 (m, 3H) 3.78 (m, 1H) 4.10 (bm, 1H) 4.93 (m, 2H) 5.14 (bs, 1H) 5.23 (bs, 1H) 5.54 (m, 1H) 6.18 (bs, 2H) 6.37 (bs, 2H).

### Example 6

### 2-(cyclopentyloxy)-9-(5-deoxy-3-C-vinyl-β-D-xylofuranosyl)-9H-purin-6-amine

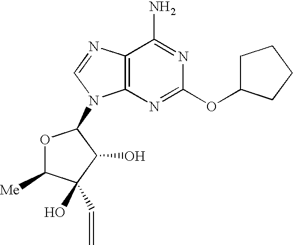

A suspension of cyclopentanol (2 ml), 9-(2-O-triisopropylsilyl-5-deoxy-3-C-vinyl-β-D-xylofuranosyl)-2-chloro-9H-purin-6-amine (300 mg, 0.64 mmol) and sodium hydroxide (256 mg, 6.4 mmol) was heated in a pressure tube at 80° C. for 36 h. After cooling to rt, excess sodium hydroxide was filtered off and washed with methylene chloride. The reaction mixture was concentrated in vacuo and the residue was purified using RP-HPLC with 10 mM ammonium acetate and acetonitrile as the mobile phases with a gradient of 5-95% in 15 min. Relevant fractions were combined to give 50 mg of the desired product.

MS (ESP): 362 (MH⁺) for C₁₇H₂₃N₅O₄

¹H NMR (400 MHz, DMSO-d₆) δ ppm 1.03 (d, 3H); 1.51-1.86 (m, 8H); 4.03 (m, 2H); 5.19-5.27 (m, 2H); 5.34-5.38 (dd, 1H); 5.60 (s, 1H); 5.70 (d, 1H); 5.98 (s, 1H); 6.0 (t, 1H); 7.17 (s, 2H); 8.02 (s, 1H).

The intermediates for this compound were prepared as follows:

### 9-(2-O-triisopropylsilyl-5-deoxy-β-D-ribofuranosyl)-2-chloro-9H-purin-6-amine

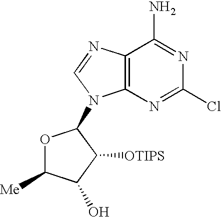

To a solution of 2-chloro-9-(5-deoxy-β-D-ribofuranosyl)-9H-purin-6-amine (5.0 g, 17.5 mmol) (see JP4046124) in dimethylformamide (70 ml) was added successively imidazole (4.77 g, 70.2 mmol) and triisopropylsilyl chloride (14.95 ml, 70.2 mmol) at rt. After stirring overnight, the reaction mixture was quenched with saturated sodium bicarbonate (5 ml) and concentrated in vacuo. The residue was dissolved in ethyl acetate (400 ml), then washed with water, dried (sodium sulfate) and concentrated to dryness. The syrup residue was purified using Gilson RP-HPLC with 10 mM ammonium acetate and acetonitrile as the mobile phases with a gradient of 50-95% in 40 min. Relevant fractions were combined to give 3.44 g of the desired product.

MS (ESP): 443 (MH⁺) for C₁₉H₃₂ClN₅O₃Si

¹H NMR (400 MHz, DMSO-d₆) δ ppm 0.93-1.04 (m, 21H); 1.37 (d, 3H); 3.93 (t, 1H); 4.06 (t, 1H); 4.96 (t, 1H); 5.13 (d, 1H); 5.86 (d, 1H); 7.84 (s, 2H); 8.39 (s, 1H).

### 9-(2-O-triisopropylsilyl-5-deoxy-β-D-erythro-pentofuranosyl-3-ulose)-2-chloro-9H-purin-6-amine

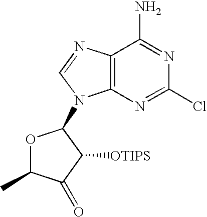

Chromium (IV) oxide (1.9 g, 19.23 mmol) was added to a solution of pyridine (3.1 ml, 38.5 mmol) in dichloromethane at 4° C., and the mixture was stirred at rt for 30 min. After addition of acetic anhydride (1.8 ml, 19.2 mmol), the mixture was stirred at rt for 30 min. A solution of 9-(2-O-triisopropylsilyl-5-deoxy-β-D-ribofuranosyl)-2-chloro-9H-purin-6-amine (1.7 g, 3.85 mmol) in dichloromethane (5 ml) was slowly added to the mixture at 4° C., and the resulting mixture was stirred overnight. The mixture was partitioned between dichloromethane (300 ml) and water (300 ml), and the organic phase was washed with water (100 ml) and brine (100 ml), dried (sodium sulfate), and evaporated. The residue was purified by chromatography over silica gel eluting with 50% ethyl acetate in hexane to give desired product (0.793 g).

MS (ESP): 441 (MH⁺) for C₁₉H₃₀ClN₅O₃Si

¹H NMR (400 MHz, DMSO-d₆) δ ppm 0.50-0.58 (m, 9H); 0.63-0.72 (m, 12H); 1.12 (d, 3H); 4.23 (qt, 1H); 5.19 (d, 1H); 5.87 (d, 1H); 7.68 (s, 2H); 8.40 (s, 1H).

### 9-(2-O— triisopropylsilyl-5-deoxy-3-C-vinyl-β-D-xylofuranosyl)-2-chloro-9H-purin-6-amine

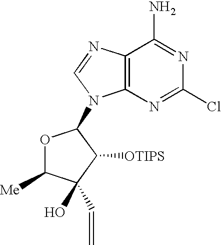

Cerium chloride (CeCl₃.7H₂O) (2.03 g, 5.45 mmol) was heated with stirring at 140° C. in vacuo (0.1 Torr) overnight and cooled. Dry tetrahydrofuran (10 ml) was added and stirring was continued for 2 h at 4° C. The resulting mixture was cooled to −78° C., and vinylmagnesium bromide (5.45 ml, 1M solution in tetrahydrofuran) was added. After stirring for 4 h at −78° C., a solution of 9-(2-O-triisopropylsilyl-5-deoxy-β-D-erythro-pentofuranosyl-3-ulose)-2-chloro-9H-purin-6-amine (0.4 g, 0.91 mmol) in tetrahydrofuran (2 ml) was added slowly and the mixture was stirred for 1 h. The orange suspension was treated with acetic acid (0.6 ml) and allowed to warm to rt. The reaction mixture was partitioned between dilute aqueous sodium bicarbonate (100 ml) and ethyl acetate (200 ml), the phases were separated, and the aqueous phase was extracted with ethyl acetate (100 ml). The combined organic phases were washed with water and brine, dried (sodium sulfate), and concentrated in vacuo. The residue was purified by chromatography over silica gel eluting with 50% ethyl acetate in hexane to give desired product (0.311 g).

MS (ESP): 469 (MH⁺) for C₂₁H₃₄ClN₅O₃Si

¹H NMR (300 MHz, DMSO-d₆) δ ppm 0.87-1.19 (m, 25H); 4.16 (dt, 1H); 4.31 (s, 1H); 5.18-5.40 (m, 1H); 5.40 (s, 1H); 5.78 (s, 1H); 6.00 (dd, 1H); 7.77 (s, 2H); 8.16 (s, 1H).

### Example 7

### 2-(cyclopentyloxy)-9-(5-deoxy-3-C-ethyl-β-D-xylofuranosyl)-9H-purin-6-amine

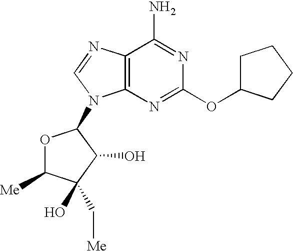

To a solution of 2-(cyclopentyloxy)-9-(5-deoxy-3-C-vinyl-β-D-xylofuranosyl)-9H-purin-6-amine (30 mg, 0.083 mmol) in ethanol (3 ml) was added 10% palladium on charcoal (50 mg). The reaction mixture was stirred under hydrogen (1 atm) for 3 h. At the end of this period, the reaction mixture was diluted with ethanol, filtered through Celite and evaporated to give desired product (30 mg).

MS (ESP): 464 (MH⁺) for C₁₇H₂₅N₅O₄

¹H NMR (400 MHz, DMSO-d₆) δ ppm 0.88 (t, 3H); 1.10 (d, 3H); 1.27 (dt, 2H); 1.50-1.85 (m, 8H); 3.83 (t, 1H); 4.01 (d, 1H); 5.17 (s, 1H); 5.23 (m, 1H); 5.62 (d, 1H); 5.83 (d, 1H); 7.15 (s, 2H); 8.01 (s, 1H).

### Example 8

### 2-(cyclopentyloxy)-9-[5-deoxy-3-C-(hydroxymethyl)-β-D-xylofuranosyl]-9H-purin-6-amine

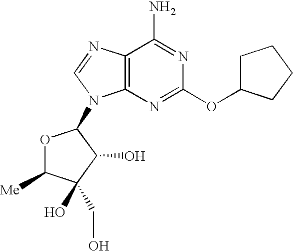

2-(Cyclopentyloxy)-9-[5-deoxy-2-O— (triisopropylsilyl)-3-C-vinyl-β-D-arabinofuranosyl]-9H-purin-6-amine (200 mg, 0.39 mmol) was dissolved in dichloromethane (4 ml) and the solution was cooled to −78° C. The reaction flask was then connected to an ozonolysis apparatus; ozone was passed through the solution for 60 min. The reaction mixture was the treated with dimethylsulfide (200 μl, 2.1 mmol) and stirred for 2 h at rt. After removal of the solvent in vacuo, the crude product was taken up in ethanol (2 ml), and a solution of sodium borohydride (30 mg, 0.8 mmol) in water (0.2 ml) was added at 4° C. The solution was stirred for 2 h at rt, diluted with ethyl acetate (50 ml), washed with water then brine, dried (sodium sulfate) and concentrated to dryness. This intermediate was taken up in tetrahydrofuran, and tetrabutylammonium fluoride (200 μl, 1M in tetrahydrofuran) and acetic acid (2 μl) were added successively. The solution was stirred for 6 h at rt and then concentrated to dryness. The residue was purified using Gilson RP-HPLC with 10 mM ammonium acetate and acetonitrile as the mobile phase with a gradient of 10-30% in 14 min. Relevant fractions were combined to give 5 mg of the desired product.

MS (ESP): 366 (MH⁺) for C₁₆H₂₃N₅O₅

¹H NMR (400 MHz, DMSO-d₆) 5 ppm 1.13 (d, 3H); 1.15-1.86 (m, 8H); 4.08 (dd, 1H); 4.28 (dd, 1H); 4.52 (t, 1H); 5.27 (s, 1H); 5.31 (m, 1H); 5.70 (d, 1H); 5.83 (d, 1H); 7.14 (s, 2H); 8.05 (s, 1H).

### Example 9

### 2-(cyclopentyloxy)-9-[5-deoxy-2-C-(hydroxymethyl)-β-D-arabinofuranosyl]-9H-purin-6-amine

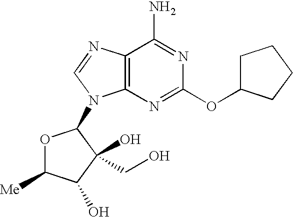

2-(cyclopentyloxy)-9-[5-deoxy-3-O-(triisopropylsilyl)-2-C-vinyl-β-D-arabinofuranosyl]-9H-purin-6-amine (138 mg, 0.267 mmol) was dissolved in dichloromethane (3 ml) and the solution was cooled to −78° C. The reaction flask was then connected to an ozonolysis apparatus; ozone was passed through the solution for 30 min. The reaction mixture was the treated with dimethylsulfide (78 μl, 0.8 mmol) and stirred for 2 h at rt. After removal solvent in vacuo, the crude product was taken up in ethanol (3 ml), and a solution of sodium borohydride (15 mg, 0.4 mmol) in water (0.2 ml) was added at 4° C. The solution was stirred for 2 h at rt, diluted with ethyl acetate (50 ml), washed with water then brine, dried (sodium sulfate) and concentrated to dryness. This intermediate was taken up in tetrahydrofuran, and tetrabutylammonium fluoride (80 μl, 1M tetrahydrofuran) and acetic acid (2 μl) were added successively. The solution was stirred for 6 h at rt, concentrated to dryness. The residue was purified using Gilson RP-HPLC with 10 mM ammonium acetate and acetonitrile as the mobile phases with a gradient of 10-95% in 40 min. Relevant fractions were combined to give 5 mg of the desired product.

MS (ESP): 366 (MH⁺) for C₁₆H₂₃N₅O₅

¹H NMR (400 MHz, DMSO-d₆) δ ppm 1.31 (d, 3H); 1.56-1.90 (m, 8H); 3.47 (dd, 1H); 3.58 (dd, 1H); 3.83 (m, 1H); 3.90 (t, 1H); 4.53 (t, 1H); 5.06 (s, 1H); 5.27 (m, 1H); 5.44 (d, 1H); 5.99 (s, 1H); 7.11 (s, 2H); 7.88 (s, 1H).

The intermediates for this compound were prepared as follows:

### 2-(cyclopentyloxy)-9-[5-deoxy-3-O-(triisopropylsilyl)-2-C-vinyl-β-D-arabinofuranosyl]-9H-purin-6-amine

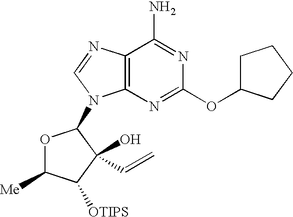

Cerium (III) chloride (CeCl₃.7H₂O) (1.40 g, 3.86 mmol) was heated with stirring at 140° C. in vacuo (0.1 Torr) for 8 h and cooled to 4° C. Dry tetrahydrofuran (5 ml) was added and stirring was continued overnight at rt. The resulting mixture was cooled to −78° C., and vinylmagnesium bromide (3.87 ml, 1M solution in tetrahydrofuran) was added. After stirring for 4 h at −78° C., a solution of 2-(cyclopentyloxy)-9-[5-deoxy-3-O-(triisopropylsilyl)-β-D-erythro-pentofuranosyl-2-ulose]-9H-purin-6-amine (0.3 g, 0.61 mmol) in tetrahydrofuran (2 ml) was added slowly via syringe over 1 h and the mixture was stirred for another 1 h. The orange suspension was treated with acetic acid (0.5 ml) and allowed to warm to rt. The reaction mixture was partitioned between dilute aqueous sodium bicarbonate (50 ml) and ethyl acetate (200 ml), the phases were separated, and the aqueous phase was extracted with ethyl acetate (100 ml). The combined organic phases were washed with water and brine, dried (sodium sulfate), and concentrated in vacuo. The residue was purified by chromatography over silica gel eluting with 50% ethyl acetate in hexane to give desired product (0.176 g).

MS (ESP): 518 (MH⁺) for C₂₆H₄₃N₅O₄Si

¹H NMR (400 MHz, DMSO-d₆) δ ppm 1.02-1.13 (m, 21H); 1.47 (d, 3H;) 1.56-1.90 (m, 8H); 4.08 (m, 2H); 5.14 (d, 1H); 5.19 (m, 1H); 5.24 (d, 1H); 5.93 (s, 1H); 6.07 (s, 1H); 6.10 (dd, 1H); 7.18 (s, 2H); 7.99 (s, 1H).

### 2-(cyclopentyloxy)-9-[5-deoxy-3-O-(triisopropylsilyl)-β-D-erythro-pentofuranosyl-2-ulose]-9H-purin-6-amine

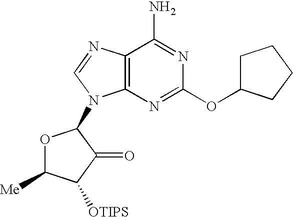

This compound was made using a procedure analogous to that used to make 9-(2-O-triisopropylsilyl-5-deoxy-β-D-erythro-pentofuranosyl-3-ulose)-2-chloro-9H-purin-6-amine by oxidation of 2-(cyclopentyloxy)-9-[5-deoxy-3-O-(triisopropylsilyl)-β-D-ribofuranosyl]-9H-purin-6-amine (described in OP-101601) with chromium (IV) oxide.

MS (ESP): 490 (MH⁺) for C₂₄H₃₉N₅O₄Si

¹H NMR (400 MHz, DMSO-d₆) δ ppm 1.08-1.10 (m, 21H); 1.48 (d, 3H); 1.64-1.89 (m, 8H); 4.09 (td, 1H); 4.76 (d, 1H); 5.20 (dq, 1H); 6.04 (s, 1H); 7.33 (s, 2H); 8.10 (s, 1H).

### Example 10

### 2-(cyclopentyloxy)-9-[3-C-(trifluoromethyl)-D-D-ribofuranosyl]-9H-purin-6-amine

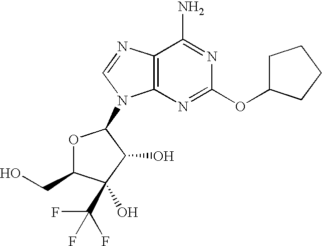

A suspension of N-[2-(cyclopentyloxy)-9H-purin-6-yl]-2,2-dimethylpropanamide (89 mg, 0.29 mmol), 1,2-di-O-acetyl-3,5-di-O-benzoyl-3-C-(trifluoromethyl)-D-ribofuranose (100 mg, 0.196 mmol), trimethylsilyl trifluoromethanesulfonate (114 μl, 0.588 mmol) and N,O-bis(trimethylsilyl)acetamide (218 μl, 0.88 mmol) in 1 ml dry acetonitrile was heated in a microwave reactor for 5 min at 130° C. The reaction mixture was quenched with triethanolamine (117 μl, 0.88 mmol) and concentrated to dryness. The residue was purified by chromatography over silica gel eluting with 50% ethyl acetate in hexane to give 9-[2-O-acetyl-3,5-di-O-benzoyl-3-C-(trifluoromethyl)-β-D-ribofuranosyl]-2-(cyclopentyloxy)-N-(2,2-dimethylpropanoyl)-9H-purin-6-amine which was taken up in 7N ammonia in methanol (5 ml). The solution was heated in a microwave reactor for 3 h at 130° C. and concentrated to dryness. The residue was purified using Gilson RP-HPLC with 10 mM ammonium acetate and acetonitrile as the mobile phases with a gradient of 5-95% in 15 min. Relevant fractions were combined to give 25 mg of the desired product.

MS (ESP): 420 (MH⁺) for C₁₆H₂₀N₅O₅F₃

¹H NMR (400 MHz, DMSO-d₆) δ ppm 1.50-1.82 (m, 8H); 3.57-3.68 (m, 2H); 3.96 (dd, 1H); 5.01 (t, 1H); 5.17-5.27 (m, 2H); 5.70 (d, 1H); 6.11 (d, 1H); 6.48 (s, 1H); 7.17 (s, 2H); 8.12 (s, 1H).

The intermediates for this compound were prepared as follows:

### 1,2-di-O-acetyl-3,5-di-O-benzoyl-3-C-(trifluoromethyl)-D-ribofuranose

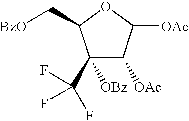

3,5-di-O-benzoyl-1,2-O-(1-methylethylidene)-3-C-(trifluoromethyl)-α-D-ribofuranose (1 g, 2.14 mmol) was dissolved in 10% aqueous trifluoroacetic acid (5 ml) and left at rt for 3 h. Solvent was evaporated to dryness and the residue was coevaporated with pyridine (2×10 ml). The residue was taken up in dry pyridine (10 ml); acetic anhydride (1.5 ml) was added to the reaction mixture. The solution was stirred overnight at rt, quenched with methanol (1 ml) and concentrated to dryness. The residue was purified by chromatography over silica gel eluting with 20% ethyl acetate in hexane to give desired product (0.96 g). 3,5-di-O-benzoyl-1,2-O-(1-methylethylidene)-3-C-(trifluoromethyl)-α-D-ribofuranose

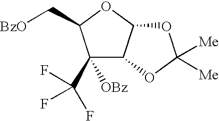

To a solution of 1,2-O(1-methylethylidene)-3-C-(trifluoromethyl)-α-D-ribofuranose (750 mg, 2.9 mmol) and 4-dimethylaminopyridine (5 mg) in dry pyridine was added slowly benzoylchloride (1.35 ml, 11.6 mmol) at 4° C. The solution was stirred for 2 days at rt. The reaction mixture was quenched with methanol (1 ml) and concentrated to dryness. The residue was purified by chromatography over silica gel eluting with 15% ethyl acetate in hexane to give desired product (1.08 g).

¹H NMR (400 MHz, DMSO-d₆) 5 ppm 1.34 (s, 3H); 1.42 (s, 3H); 4.60-4.68 (m, 1H); 4.78-4.85 (m, 2H); 5.50 (d, 1H); 6.06 (d, 1H); 7.54 (dt, 4H); 7.67-7.76 (m, 2H); 8.03 (d, 4H).

### 1,2-O-(1-methylethylidene)-3-C-(trifluoromethyl)-α-D-ribofuranose

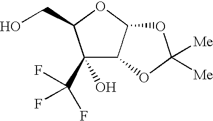

3-C-Trifluoromethyl-1,2:5,6-di-O-isopropylidene-α-D-allofaranose (1.1 g, 3.35 mmol) (made as described in Tetrahedron: Asymmetry, (1998), 9, 213-226) was dissolved in 80% aqueous acetic acid (20 ml) and left at rt for 40 h. Solvent was evaporated to dryness and the residue was taken up in ethanol (10 ml). A solution of sodium metaperiodate (0.86 g, 4.01 mmol) in water (10 ml) was added to the reaction mixture with stirring. Thick slurry formed and stirring was continued for 40 min. The precipitates were filtered off and washed with ethanol. The filtrates were combined and concentrated to dryness. Ethanol (70 ml) was added to the residue, and the precipitate was filtered off. The residue was dissolved in ethanol (30 ml), sodium borohydride (116 mg, 3.05 mmol) was added and the solution was stirred overnight at rt. The reaction mixture was concentrated in vacuo and partitioned between ethyl acetate (200 ml) and water (100 ml). The organic phase was separated, washed with brine, dried over sodium sulfate, and concentrated to dryness to give desired product (0.76 g).

¹H NMR (400 MHz, DMSO-d₆) δ ppm 1.31 (s, 3H); 1.9 (s, 3H); 3.45-3.50 (m, 1H); 3.60-3.69 (m, 1H); 4.04 (dd, 2.02 Hz, 1H); 4.55 (d, 1H); 4.96 (t, 1H); 5.79 (d, 1H); 6.70 (s, 1H).

### N-[2-(cyclopentyloxy)-9H-purin-6-yl]-2,2-dimethylpropanamide

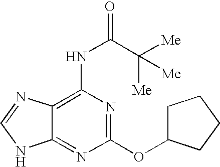

To a solution of 2-(cyclopentyloxy)-9-(tetrahydrofuran-2-yl)-9H-purin-6-amine (12.27 g, 42.46 mmol) in pyridine (50 ml) was added pivaloyl chloride (8.9 ml, 67.9 mmol) and 4-dimethylaminopyridine (56.0 mg, 0.42 mmol) at 5° C. After stirring overnight at rt, the reaction mixture was concentrated in vacuo. The residue was dissolved in dichloromethane (120 ml), and then washed with water, saturated sodium bicarbonate, brine, dried (sodium sulfate) and concentrated to dryness. The residue was taken up in dichloromethane (120 ml), and trifluoroacetic acid (80 ml) was added slowly at 5° C. The solution was stirred for 3 h at rt and concentrated to dryness. The residue was purified using flash chromatography (4:1 ethyl acetate/hexanes). Relevant fractions were combined to give 28.7 g of the desired product.

MS (ESP): 304 (MH⁺) for C₁₅H₂₁N₅O₂

¹H NMR (400 MHz, DMSO-d₆) 5 ppm 1.3 (s, 9H); 1.5-1.8 (m, 7H); 1.8-1.9 (m, 2H); 5.4 (m, 1H); 8.5 (s, 1H); 10.6 (s, 1H).

### Example 11

### 2-(cyclopentyloxy)-9-[5-deoxy-5-fluoro-3-C-(trifluoromethyl)-3-D-ribofuranosyl]-9H-purin-6-amine

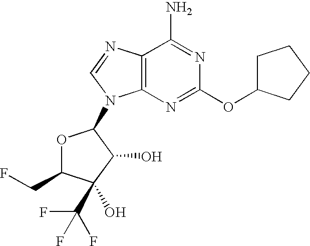

To a solution of 2-(cyclopentyloxy)-9-[3-C-(trifluoromethyl)-β-D-ribofuranosyl]-9H-purin-6-amine (65 mg, 0.155 mmol) in dry pyridine (2 ml) was added tosyl chloride (80 mg, 0.41 mmol) at 4° C. The solution was stirred at 4° C. for 2 days, and then diluted with dichloromethane (50 ml). The reaction mixture was washed with saturated sodium bicarbonate and the aqueous phase was extracted with dichloromethane (50 ml). The organic phases were combined and concentrated in vacuo. The residue was taken up in tetrabutylammonium fluoride (1 ml, 1M tetrahydrofuran), and the solution was heated in a microwave reactor for 1 h at 100° C. and concentrated to dryness. The residue was purified using Gilson RP-HPLC with 10 mM ammonium acetate and acetonitrile as the mobile phase with a gradient of 5-95% in 15 min. Relevant fractions were combined to give 25 mg of the product, which was re-purified using Gilson RP-HPLC with 10 mM ammonium acetate and acetonitrile as the mobile phase with a gradient of 25-30% in 15 min. Relevant fractions were combined to give 1.7 mg of the desired product.

MS (ESP): 422 (MH⁺) for C₁₆H₁₉F₄N₅O₄

¹H NMR (400 MHz, MeOD) δ ppm 1.59-1.88 (m, 8H); 4.31 (m, 1H); 4.61 (m, 1H); 4.77 (m, 1H); 5.30 (m, 2H); 5.93 (d, 1H); 8.05 (s, 1H).

### Example 12

### (3S,4R,5R)-5-[6-amino-2-(cyclopentyloxy)-9H-purin-9-yl]-2,2-bis(hydroxymethyl)tetrahydrofuran-3,4-diol

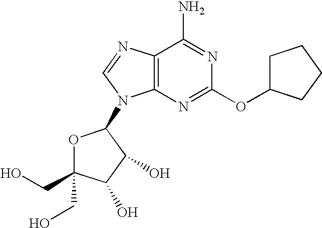

A mixture of {(3aS,6R,6aR)-6-[6-amino-2-(cyclopentyloxy)-9H-purin-9-yl]-2,2-dimethyltetrahydrofuro[3,4-d][1,3]dioxole-4,4-diyl}dimethanol and {(3aS,6R,6aR)-6-[6-amino-2-(cyclopentyloxy)-9H-purin-9-yl]tetrahydrofuro[3,4-d][1,3]dioxole-4,4-diyl}dimethanol obtained as described below was dissolved in a 2:1 mixture of formic acid/water and stirred at rt for 15 h. Solvents were then evaporated to dryness. The resulting crude mixture was purified by RP-HPLC (ammonium acetate/acetonitrile pH 8). After combining the relevant fractions, evaporating solvents, re-dissolving in water/methanol and lyophilizing, the desired product was obtained as a white solid (8.0 mg, >95% pure).

MS (ESP): 382 (MH⁺) for C₁₆H₂₃N₅O₆.

¹H NMR (300 MHz, DMSO-d₆) δ ppm 1.70 (m, 4H) 1.85 (m, 6H) 3.56 (m, 4H) 4.17 (d, 1H) 4.79 (m, 1H) 5.29 (m, 1H) 5.75 (d, 1H) 7.23 (bs, 2H) 8.11 (s, 1H). The intermediates were prepared as follows:

### {(3aS,6R,6aR)-6-[6-amino-2-(cyclopentyloxy)-9H-purin-9-yl]-2,2-dimethyltetrahydrofuro[3,4-d][1,3]dioxole-4,4-diyl}dimethanol and

### {(3aS,6R,6aR)-6-[6-amino-2-(cyclopentyloxy)-9H-purin-9-yl]tetrahydrofuro[3,4-d][1,3]dioxole-4,4-diyl}dimethanol

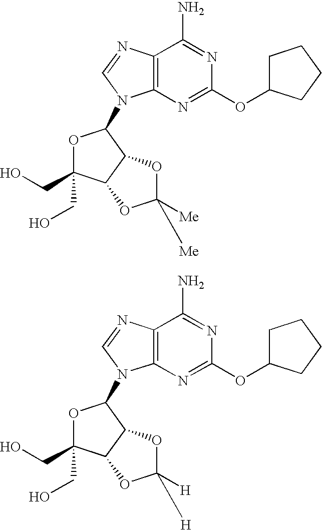

{(3aS,4S,6R,6aR)-6-[6-amino-2-(cyclopentyloxy)-9H-purin-9-yl]-2,2-dimethyltetrahydrofuro[3,4-d][1,3]dioxol-4-yl}methanediol hydrate (400 mg, 1.0 mmol) was dissolved in 7 mL of dioxane at rt. Formaldehyde (35% in water, 0.6 mL) was then added, followed by 2 mL of 1N sodium hydroxide. The mixture was stirred at rt for 10 min and then neutralized by slow addition of acetic acid to pH 6-7. Solvents were evaporated to dryness and the crude mixture was partitioned between chloroform and water. The organic layer was separated and dried over magnesium sulfate. Solvents were evaporated to dryness and this crude mixture was re-dissolved in ethanol (10 mL). After cooling to 0° C., sodium borohydride (40 mg, 1.0 mmol, 1.0 eq) was added in one portion, and the mixture was stirred for 20 min at the same temperature. The reaction was quenched by slow addition of acetic acid to pH 6-7 and the solvents were evaporated under vacuum. The resulting crude mixture was separated by flash chromatography on an ISCO system (dichloromethane/methanol gradient 0 to 20% over 1 h).

MS (ESP): 422 (MH⁺) for C₁₉H₂₇N₅O₆ in >90% purity.

¹H NMR (300 MHz, CDCl₃) 5 ppm 1.34 (s, 1H) 1.60 (s, 3H) 1.60 (m, 4H) 1.90 (m, 6H) 3.81 (m, 4H) 5.09 (m, 1H) 5.28 (m, 1H) 5.43 (m, 1H) 5.82 (d, 1H) 6.11 (bs, 1H) 7.64 (s, 1H).

### {(3aS,6R,6aR)-6-[6-amino-2-(cyclopentyloxy)-9H-purin-9-yl]tetrahydrofuro[3,4-d][1,3]dioxole-4,4-diyl}dimethanol

MS (ESP): 394 (MH⁺) for C₁₇H₂₃N₅O₆.

### {(3aS,4S,6R,6aR)-6-[6-amino-2-(cyclopentyloxy)-9H-purin-9-yl]-2,2-dimethyltetrahydrofuro[3,4-d][1,3]dioxol-4-yl}methanediol

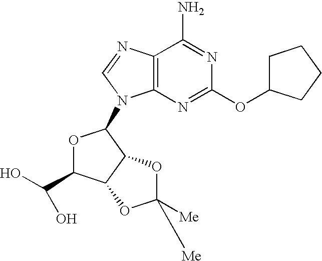

2-(cyclopentyloxy)-9-[2,3-O-(1-methylethylidene)-β-D-ribofuranosyl]-9H-purin-6-amine (1.0 g, 2.6 mmol), was dissolved under nitrogen in 100 mL anhydrous dichloromethane. The solution was cooled to 0° C. and added dropwise to a freshly prepared solution of Dess-Martin periodinane (1.87 g, 4.42 mmol, 1.7 eq) in 100 mL dichloromethane and 5 mL dimethylsulfoxide. After 10 min of stirring at 0° C., the mixture was allowed to reach rt and stirred for 30 min. At this point the reaction mixture was partitioned between a solution of saturated sodium bicarbonate/saturated sodium thiosulfate (100 mL) and chloroform (150 mL). After shaking vigorously for 3 min, the organic layer was separated and dried over magnesium sulfate. The solution was filtered and the solvent evaporated under vacuum to give a thick yellow oil. Water was added and a white precipitate formed. This was collected by filtration and air-dried overnight giving a quantitative yield of desired product. Due to its inherent instability in solution, this product was only characterized by LC/MS for a small amount dissolved in acetic acid.

MS (ESP): 408 (MH⁺) for C₁₈H₂₅N₅O₆

### Example 13

### (3S,4R,5R)-5-[6-amino-2-(cyclopentyloxy)-9H-purin-9-yl]-2,2-bis(fluoromethyl)tetrahydrofuran-3,4-diol

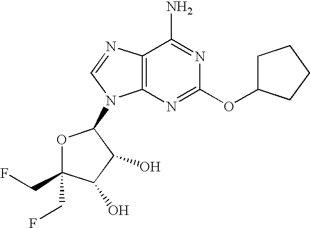

The mixture containing [(2S,3S,4R,5R)-5-[6-amino-2-(cyclopentyloxy)-9H-purin-9-yl]-2-(fluoromethyl)-3,4-dihydroxytetrahydrofuran-2-yl]methyl 4-methylbenzenesulfonate, was dissolved in 2 mL of dry tetrahydrofuran and 1.5 mL of tetrabutylammonium fluoride (1M in tetrahydrofuran) in a microwave vial with stirring. The mixture was heated in a microwave reactor at 120° C. for 1 h. Solvents were evaporated to dryness and the crude partitioned between ethyl acetate and water and worked up using standard methods. The desired final product was isolated by RP-HPLC using an ammonium acetate/acetonitrile (pH 8) gradient. After combining relevant fractions and lyophilizing the product was obtained as an off-white solid (5.3 mg, 80% pure).

MS (ESP): 386 (MH⁺) for C₁₆H₂₁F₂N₅O₄.

¹H NMR (300 MHz, DMSO-d₆) δ ppm 1.60 (1H, 4H) 1.70 (m, 4H) 1.90 (m, 2H) 3.86 (d, 1H) 4.01 (d, 1H) 4.50 (m, 2H) 4.70 (m, 1H) 4.90 (d, 1H) 5.29 (m, 1H) 5.82 (s, 1H) 7.25 (bs, 2H) 8.0 (s, 1H).

The intermediates for this compound were prepared as follows:

### [(2S,3S,4R,5R)-5-[6-amino-2-(cyclopentyloxy)-9H-purin-9-yl]-2-(fluoromethyl)-3,4-dihydroxytetrahydrofuran-2-yl]methyl 4-methylbenzenesulfonate

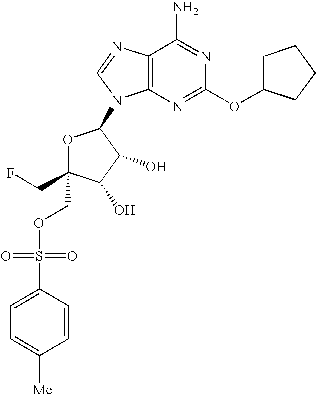

The mixture of fluorinated intermediates, prepared as described below, was dissolved in 2:1 formic acid/water and stirred at rt for 4 days. Analysis of the mixture by LC/MS showed the presence of two major products corresponding to the acetonide deprotected versions of the major components of the starting material mixture. Solvents were evaporated to dryness and the resulting crude mixture used in the final step without additional purification.

MS (ESP): 538 (MH⁺) for C₂₃H₂₈FN₅O₇S

### Preparation of Mixture of Fluorinated Intermediates.

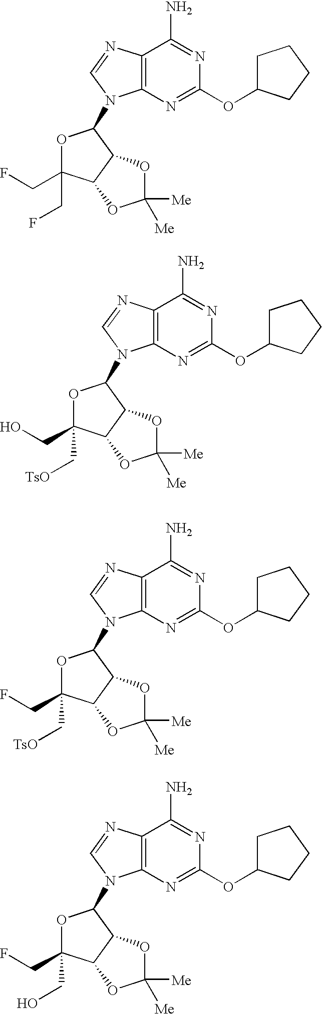

The mixture of tosylated intermediates, prepared as described below, was dissolved in 3 mL of dry tetrahydrofuran and 1.4 mL of tetrabutylammonium fluoride (1M in tetrahydrofuran, 4 eq) in a microwave vial. The mixture was heated using a microwave reactor at 120° C. for 2 h. Solvents were evaporated to dryness to give a brown oil. This was shown by LC/MS analysis to be a mixture of at least 4 products (difluorinated, mono fluoro-mono OH, mono tosyl-mono OH, and mono tosyl-mono fluoro). This crude mixture was used in the next step without additional purification.

MS (ESP): 576 (MH⁺) for C₂₆H₃₃N₅O₈S. minor

MS (ESP): 424 (MH⁺) for C₁₉H₂₆FN₅O₅. major

MS (ESP): 426 (MH⁺) for C₁₉H₂₅F₂N₅O₄. very minor

MS (ESP): 578 (MH⁺) for C₂₆H₃₂FN₅O₈S. major

### Preparation of Mixture of Tosylated Intermediates.

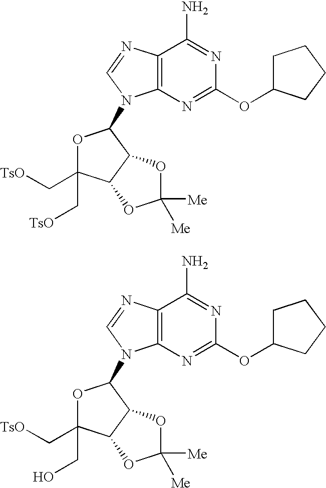

{(3aS,6R,6aR)-6-[6-amino-2-(cyclopentyloxy)-9H-purin-9-yl]-2,2-dimethyltetrahydrofuro[3,4-d][1,3]dioxole-4,4-diyl}dimethanol (155 mg, 0.35 mmol) was dissolved in 10 mL of dry pyridine under nitrogen. The resulting solution was cooled to 0° C. and tosyl chloride (105 mg, 0.55 mmol, 1.5 eq) was added in one portion. The mixture was stirred at 0° C. for 2 h and then left standing in freezer (−20° C.) for 15 h. Next, 0.5 eq of tosyl chloride were added and the mixture placed in freezer for an additional 15 h. The reaction mixture was then evaporated to dryness and partitioned between ethyl acetate and saturated sodium bicarbonate. After standard work-up an off-white solid was obtained. This was shown by LC/MS analysis to be a mixture of at least three products (two mono-tosylated products and one bis-tosylated). This mixture was directly used in the next step without additional purification.

MS (ESP): 576 (MH⁺) for C₂₆H₃₃N₅O₈S. minor

MS (ESP): 576 (MH⁺) for C₂₆H₃₃N₅O₈S. major

MS (ESP): 730 (MH⁺) for C₃₃H₃₉N₅O₁₀S₂. major

### Example 14

### (2S,3S,4R,5R)-5-[6-amino-2-(cyclopentyloxy)-9H-Purin-9-yl]-2-(iodomethyl)-2-methoxytetrahydrofuran-3,4-diol

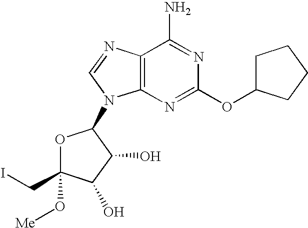

A solution of 2-(cyclopentyloxy)-9-[(3aR,4R,6S,6aS)-6-(iodomethyl)-6-methoxy-2,2-dimethyltetrahydrofuro[3,4-d][1,3]dioxol-4-yl]-9H-purin-6-amine in 2:1 formic acid/water was stirred for 2 days at rt. The reaction mixture was concentrated to dryness and purified by RP-HPLC with 10 mM ammonium acetate and acetonitrile as the mobile phase.

MS (ESP): 492 (MH⁺) for C₁₆H₂₂IN₅O₅

¹H NMR (300 MHz DMSO-d₆) δ ppm 0.9 (m, 1H) 1.22 (m, 2H) 1.69 (m, 3H) 1.95 (m, 2H) 3.5 (m, 2H) 4.5 (s, 1H) 4.8 (s, 1H) 5.1 (s, 1H) 5.2 (s, 1H) 5.5 (s, 1H) 5.8 (s, 1H) 7.2 (s, 2H) 8.1 (s, 1H).

The intermediates for this compound were prepared as follows:

### 2-(cyclopentyloxy)-9-[(3aR,4R,6S,6aS)-6-(iodomethyl)-6-methoxy-2,2-dimethyltetrahydrofuro[3,4-d][1,3]dioxol-4-yl]-9H-purin-6-amine

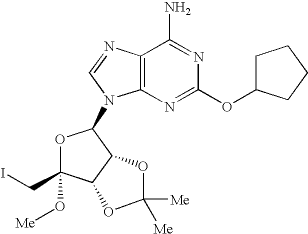

2-(cyclopentyloxy)-9-[5-deoxy-2,3-O-(1-methylethylidene)-(3-D-erythro-pent-4-enofuranosyl]-9H-purin-6-amine (300 mg, 0.8 mmol) was dissolved in methanol. Silver acetate (801 mg, 4.8 mmol) was added. A solution of iodine in methanol (508 mg in 15 mL) was added dropwise over 30 min. Reaction was stirred an additional 30 min. The reaction mixture was quenched with 10% sodium thiosulfate/sodium bicarbonate, filtered over Celite and taken up in ethyl acetate. The organic layer was extracted with sodium bicarbonate and brine, dried over magnesium sulfate, and concentrated. The product was used without further purification or characterization.

### 2-(cyclopentyloxy)-9-[5-deoxy-2,3-O-(1-methylethylidene)-β-D-erythro-pent-4-enofuranosyl]-9H-purin-6-amine

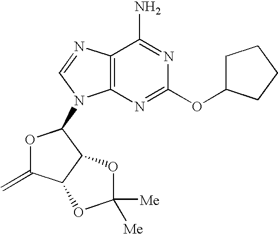

2-(cyclopentyloxy)-9-[5-deoxy-5-iodo-2,3-O-(1-methylethylidene)-D-ribofuranosyl]-9H-purin-6-amine was taken up in dry tetrahydrofuran. An excess of potassium tert-butoxide was added and the reaction mixture was stirred for 30 min at rt. Sodium bicarbonate was added to quench reaction. Ethyl acetate was added and the layers were separated. Organic layer was washed with brine and dried over magnesium sulfate.

### 2-(cyclopentyloxy)-9-[5-deoxy-5-iodo-2,3-O-(1-methylethylidene)-D-ribofuranosyl]-9H-purin-6-amine

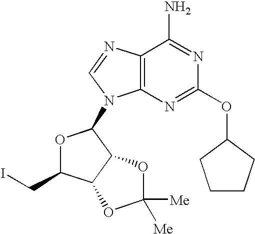

2-(cyclopentyloxy)-9-[2,3-O-(1-methylethylidene)-D-ribofuranosyl]-9H-purin-6-amine was dissolved in dimethylformamide and cooled to 0° C. Methyl triphenoxyphosphonium iodide (2.5 eq) was added and stirred at rt overnight. Ethyl acetate was added to the reaction mixture and was washed with sodium bicarbonate and brine. The reaction mixture was concentrated and the crude product was used for the next reaction.

### Example 15

### 2-[6-amino-2-(cyclopentyloxy)-9H-purin-9-yl]-6-deoxy-6-fluoro-β-D-psicofuranosyl

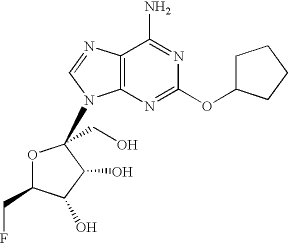

A suspension of N-[2-(cyclopentyloxy)-9H-purin-6-yl]benzamide (32 mg, 0.99 mmol), 1,2,3,4-tetra-O-benzoyl-6-deoxy-6-fluoro-D-psicofuranose (770 mg, 1.28 mmol) and N,O-bis (trimethylsilyl)acetamide (733 μl, 2.98 mmol) in 15 ml dry acetonitrile was warmed to 60° C. After stirring for 30 min, 355 μl (2.98 mmol) tin tetrachloride was added dropwise at rt and stirring was continued for another 2 h at 60° C. The reaction mixture was cooled to room temperature and poured into a mixture of cold saturated sodium bicarbonate and dichloromethane (1:1, v/v, 200 ml). The aqueous phase was extracted with dichloromethane (50 ml). The organic phases were combined and washed with saturated sodium bicarbonate, dried (sodium sulfate) and evaporated to dryness. This intermediate (219 mg) was taken up in 7N ammonia in methanol (5 ml) at rt and the stirring was continued for 1 h. The reaction mixture was concentrated in vacuo and the residue was dissolved in a mixture of methanol/THF 1:1 (10 ml), a solution of 1N sodium hydroxide (2 ml) was added. After stirring overnight at rt, the reaction mixture was neutralized with amberlite IR-120⁺, filtered and concentrated in vacuo. The residue was purified using Gilson reverse phase HPLC with 10 mM ammonium acetate and acetonitrile as the mobile phases with a gradient of 0-95% in 15 min. Relevant fractions were combined to give 12.1 mg of the desired product.

MS (ESP): 384 (MH⁺) for C₁₅H₂₂FN₅O₅

¹H NMR (400 MHz, DMSO-D6) δ ppm 1.50-1.87 (m, 8H) 3.77-3.80 (s, 2H) 3.98-4.20 (m, 2H) 4.42-4.70 (m, 3H) 4.93 (m, 1H) 5.18-5.22 (m, 2H) 5.68 (d, 1H) 7.03 (s, 2H) 7.72 (s, 1H).

The intermediates for this compound were prepared as follows:—

### 1,2,3,4-tetra-O-benzoyl-6-deoxy-6-fluoro-D-psicofuranose

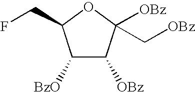

A solution of 1,2,3,4-di-O-isopropylidene-6-O-p-toluenesulfonyl-β-D-psicofuranose (3.05 g, 7.37 mmol) and tetrabutylammonium fluoride (20 ml, 1M solution in THF) was heated under pressure for 3 h at 120° C. The reaction mixture was diluted with diethyl ether (250 ml) at rt, washed with water, dried (sodium sulfate), and concentrated in vacuo. The residue was taken up in acetic acid (50 ml, 70% in water) and heated at 100° C. for 4 h. The solution was concentrated in vacuo and the residue was co-evaporated to dryness with toluene (2×10 ml). This residue was treated with dry pyridine (50 ml). Benzoyl chloride (8 ml) was added at 4° C., and the stirring was continued over weekend. After adding methanol (1 ml), the reaction mixture was concentrated in vacuo. The residue was dissolved in dichloromethane, then washed with water, saturated sodium bicarbonate and brine, dried (sodium sulfate), and concentrated in vacuo. The residue was purified by chromatography over silica gel eluting with 25% ethyl acetate in hexane to give desired product (2.7 g).

MS (ESP): 599 (MH⁺) for C₃₄H₂₇FO₉

### 1,2:3,4-Di-O-isopropylidene-6-O-p-toluenesulfonyl-D-D-psicofuranose

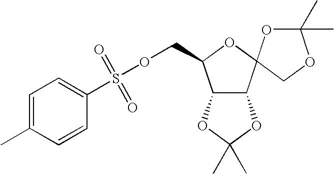

A solution of 1,2,3,4-di-O-isopropylidene-β-D-psicopyranose (3 g, 11.5 mmol) and sulfuric acid (200 μl) in acetone (75 ml) was stirred at rt for 27 h. After adding ammonium hydroxide (5 ml), the reaction mixture was filtered and the filtrate was concentrated in vacuo. The residue was dissolved in diethyl ether (200 ml), washed with water (2×30 ml), dried (sodium sulfate), and concentrated to dryness. This residue was treated with p-toluenesulfonyl chloride (3.3 g, 17.3 mmol) in pyridine (15 ml) at rt for 24 h. The reaction mixture was quenched with water (1 ml) and concentrated to dryness. The residue was dissolved in diethyl ether (150 ml), washed with water (100 ml), dried (sodium sulfate), and concentrated in vacuo to give desired product as a beige solid (3.14 g).

¹H NMR (300 MHz, DMSO-D6) δ ppm 1.20-1.27 (m, 9H) 1.33 (s, 3H) 2.42 (s, 3H) 3.87-4.01 (m, 2H) 4.05-4.18 (m, 3H) 4.56 (d, 1H) 4.71 (d, 1H) 7.49 (d, 2H) 7.79 (d, 2H)

### Example 16

### (2R,3S,4R,5R)-2-(6-amino-2-(cyclopentyloxy)-9H-purin-9-yl)-5-methyl-3-vinyltetrahydrofuran-3,4-diol AZ12495498

(2R,3S,4R,5R)-2-(6-amino-2-(cyclopentyloxy)-9H-purin-9-yl)-5-methyl-3-vinyltetrahydrofuran-3,4-diol may be prepared by heating cyclopentanol, sodium hydroxide and (2R,3S,4R,5R)-2-(6-amino-2-chloro-9H-purin-9-yl)-5-methyl-3-vinyltetrahydrofuran-3,4-diol in a pressure tube at 80° C. After cooling to rt, excess sodium hydroxy may be filtered off and wash with methylene chloride. The product may be obtained by concentrating the reaction mixture in vacuo.

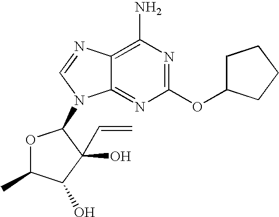

The intermediates for this compound may similarly be prepared as in Example 6.

### Reference Compound

### 6-amino-2-(pentylthio)-9-(3-D-ribofuranosyl-9H-purin-8-ol

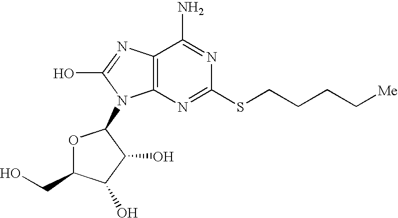

6-{[(1E)-(Dimethylamino)methylene]amino}-2-(pentylthio)-9-(2,3,5-tri-O-benzoyl-β-D-ribofuranosyl)-9H-purin-8-ol (150 mg, 0.2 mmol) was dissolved in ammonia (5 mL, 7 N in methanol) to which aqueous sodium hydroxide (3 mL, 1 N) was added. The reaction was then stirred at rt for 16 h, followed by concentration in vacuo. The crude product was purified by RP-HPLC. Yield 1 mg (1.3%)

MS (APCI-pos) obs M+H @ 386 amu, M−H (383.6 amu

¹H NMR (CD₃OD): 5.79 (d, 1H); 4.23 (dd, 1H); 3.95 (m, 1H), 3.55-3.74 (q, 2H), 3.44 (dd, 1H); 3.29 (m, 2H); 3.15 (s, 2H); 2.95-3.1 (m, 4H); 1.61 (t, 2H); 1.30 (m, 6H).

The intermediates for this compound were prepared as follows:—

### 2-(pentylthio)-pyrimidine-4,6-diamine

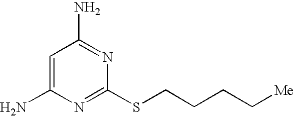

Pyrimidine-4,6-diamine-2-thiol (10 g, 70.3 mmol) was dissolved in methanol (140 mL) at rt to which 70 mL of 1N sodium hydroxide was added. This was followed by n-pentyl bromide (8.5 mL, 1.1 eq) and 100 mL dimethylformamide. The reaction was then stirred at rt for 18 h. LC/MS indicated clean conversion to the desired product, and consumption of starting material. The reaction was then concentrated in vacuo to provide an oil. The crude reaction mixture was then diluted with water (200 mL) and extracted with dichloromethane (3×300 mL). The organic portions were combined and dried over sodium sulfate, filtered and concentrated in vacuo. The product was used without further purification; yield 14.8 g (69.7 mmol)

### 5-nitroso-2-(pentylthio)-pyrimidine-4,6-diamine

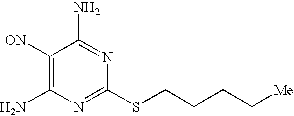

2-(Pentylthio)-pyrimidine-4,5,6-triamine (14.8 g, 69.7 mmol) was dissolved in glacial acetic acid (280 mL) and water (60 mL) followed by cooling to 0° C. Sodium nitrite (9 g, 1.9 eq) was prepared as a solution in water (60 mL) and cooled to 0° C., separately. The sodium nitrite solution was added dropwise over 10 min to the solution of the diaminopyrimidine. Upon addition a dark solid forms. The reaction was then stirred at 0° C. for 1.5 h. The solids were isolated by vacuum filtration and washed with ice water to yield a dark purple solid. The solids were dried under high vacuum for two days. Yield: 11.8 g (48.9 mmol, 70.1%).

MS (APCI-pos) obs M+H (242.5 amu

### 2-(pentylthio)-pyrimidine-4,5,6-triamine

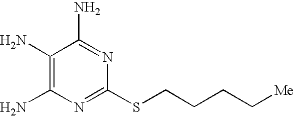

5-Nitroso-2-(pentylthio)-pyrimidine-4,6-diamine (1 g, 4.1 mmol) was suspended in ethanol (21 mL) at rt. This was followed by the addition of glacial acetic acid (1.6 mL) and zinc turnings (1.4 g, 21.5 mmol). The reaction was then heated at 70° C. for 18 h. Little reaction was observed, so the temperature was then raised to 85° C. and LC/MS confirmed consumption of the starting material within 3 h. The reaction was then cooled to rt and filtered through a glass fiber filter, washing with 70 mL ethanol. The crude reaction mixture was then concentrated in vacuo. The crude product was purified by flash chromatography, eluting with 0 to 6.25% methanol:dichloromethane. Yield 296 mg (1.3 mmol, 33%) as a pale colored oil.

MS (APCI-pos) obs M+H @ 228.1 amu

### 6-amino-2-(pentylthio)-9H-purin-8-ol

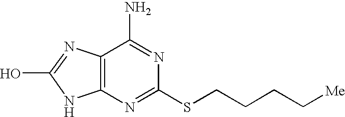

2-(Pentylthio)-pyrimidine-4,5,6-triamine (147 mg, 0.65 mmol) was dissolved in dichloromethane (2 mL) at rt. This was followed by addition of triethylamine (0.5 mL) and then triphosgene (220 mg, 1.1 eq). After 30 min LC/MS indicated greater than 90% conversion to the desired product. After 3 h the reaction was quenched with methanol (1 mL) and the reaction concentrated in vacuo. The product was used without further purification.

### N′-[8-hydroxy-2-(pentylthio)-9H-purin-6-yl]-N,N-dimethylimidoformamide

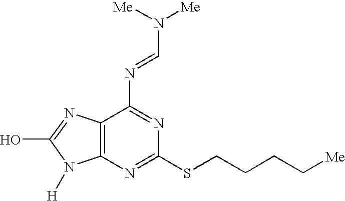

6-amino-2-(pentylthio)-9H-purin-8-ol was partially dissolved in dimethylformamide (2 mL). This was followed by the addition of dimethylformamide dimethyl acetal (0.5 mL). The reaction was then stirred at rt for 18 h, followed by concentrating in vacuo. LC/MS indicated that the conversion to the imine was ˜30% complete. The crude reaction mixture was then resubjected to dimethylformamide dimethyl acetal, with 6 mL acetonitrile to help dissolve the remaining starting material. After 2 d heating at 60° C. the reaction was concentrated, LC/MS analysis indicated nearly complete conversion to the protected product.

MS (APCI-pos) obs M+H (309.1 amu

### 6-{[(1E)-(dimethylamino)methylene]amino}-2-(pentylthio)-9-(2,3,5-tri-O-benzoyl-β-D-ribofuranosyl)-9H-purin-8-ol

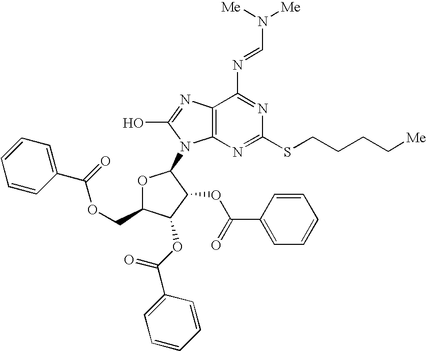

N′-[8-hydroxy-2-(pentylthio)-9H-purin-6-yl]-N,N-dimethylimidoformamide (150 mg, 0.48 mmol) was dissolved in acetonitrile (10 mL) at rt, followed by addition of 1-O-acetyl-2,3,5-tri-O-benzoyl-D-ribofuranose (1 g, 4.56 eq), and N,N-bis(trimethylsilyl)acetamide (1 mL, 8.9 eq). After stirring for 30 min at 60° C. the reaction was cooled to rt, followed by the addition of neat tin (IV) chloride (0.6 mL, 6.6 eq). The reaction was then heated to 65° C. overnight under nitrogen. The reaction was then cooled to rt and concentrated in vacuo. The crude reaction was resuspended in dichloromethane (50 mL) and washed by saturated sodium bicarbonate (2×200 mL) and brine (2×200 mL). The organic portions were then dried over magnesium sulfate, filtered and concentrated in vacuo. The crude product was purified by RP-HPLC. Yield 150 mg (40%).

MS (APCI-pos) obs M+H (753.3 amu

1. A compound of formula I 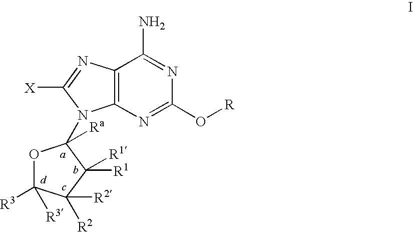
in free or salt form, wherein:
X is hydrogen, halo, NR⁸R⁹, azido, cyano, isocyano, hydroxy, C₁₋₆alkyl, C₂₋₆alkenyl, C₂₋₆alkynyl, aryl, C₃₋₈cycloalkyl, C₃₋₈cycloalkenyl, heterocyclyl, —OR⁷, —C(O)R⁵, —OC(O)R⁵, S(O)ₚR⁴, —NHC(O)NR⁸R⁹, —N(C₁₋₆alkyl)C(O)NR⁸R⁹, —NHC(O)R⁷, —NHCO₂R⁷, —NHSO₂(R⁴), -amidino i.e. —NHC(NH)NH₂; provided that when X is hydrogen, at least one carbon selected from C-a, C-b, C-c, and C-d is quaternary;
R is selected from C₁₋₈alkyl, C₂₋₈alkenyl, C₂₋₈alkynyl, C₃₋₈carbocyclyl, aryl, and heterocyclyl, any of which may be optionally substituted on one or more carbon atom by R′;
p is independently at each occurrence 0, 1 or 2;
R^{a} is hydrogen, C₁₋₆alkyl, C₂₋₆alkenyl, hydroxy(C₁₋₆alkyl), and cyano, any of which may be optionally substituted on one or more carbon atom by R′;
R¹, R^{1′}, R², R^{2′}, R³, and R^{3′} are each independently selected from hydrogen, hydroxy, cyano, azido, C₁₋₆alkyl, C₃₋₈carbocyclyl, halo, —C(O)R⁵, —OC(O)R⁵, S(O)ₚR⁴, C₂₋₆alkenyl, C₂₋₆alkynyl, heterocyclyl, —OR⁷, NR⁸R⁹, wherein R¹, R^{1′}, R², R^{2′}, R³, and R^{3′} may be optionally substituted on one or more carbon atoms by one or more R; or alternatively,
R¹ and R^{1′}, R² and R^{2′}, or R³ and R^{3′}, taken together with the carbon to which they are attached, form C═O or C═N—O—R⁶, or an optionally substituted 3, 4, 5, 6, or 7-membered ring containing 0, 1, or 2 heteroatoms selected from O, S, NH, or N(C₁₋₆alkyl); or alternatively,
R¹ and R², R² and R³, taken together with the carbons to which they are attached, form an optionally substituted 3, 4, 5, or 6-membered ring containing 0, 1, or 2 heteroatoms selected from O, S, NH, or N(C₁₋₆alkyl);
R⁴ at each occurrence is independently —NR⁸R⁹, C₁₋₆alkyl, C₂₋₆alkenyl, C₁₋₆alkoxy, C₃₋₈cycloalkyl, heterocyclyl, and aryl wherein R⁴ may be optionally substituted on one or more carbon atoms by one or more R;
R⁵ at each occurrence is independently hydrogen, —NR⁸R⁹, —OR⁷, C₁₋₆alkyl, C₂₋₆alkenyl, C₃₋₈cycloalkyl, C₃₋₈cycloalkenyl, heterocyclyl, and aryl wherein each R⁵ may be optionally substituted on one or more carbon atoms by one or more R;
R⁶ at each occurrence is independently hydrogen, C₁₋₆alkyl, C₂₋₆alkenyl, C₂₋₆alkynyl, C₃₋₈cycloalkyl, C₃₋₈cycloalkenyl, heterocyclyl and aryl, wherein each R⁶ may be optionally substituted on one or more carbon atoms by one or more R;
R⁷ at each occurrence is independently hydrogen, C₁₋₆alkyl, C₂₋₆alkenyl, C₃₋₈cycloalkyl, C₃₋₈cycloalkenyl, aryl, S(O)ₚR⁴, and heterocyclyl wherein R⁷ may be optionally substituted on one or more carbon by one or more R′;
R⁸ and R⁹ are each independently selected from hydrogen, C₁₋₆alkyl, C₂₋₆alkenyl, C₂₋₆alkynyl, —OR⁷, C₃₋₈cycloalkyl, C₃₋₈cycloalkenyl, heterocyclyl, and aryl, wherein each R⁸ or R⁹ may be optionally substituted on one or more carbon atoms by one or more R;
R′ at each occurrence is independently halo, hydroxy, nitro, —NR⁸R⁹, azido, cyano, isocyano, C₁₋₆alkyl, C₂₋₆alkenyl, C₂₋₆alkynyl, aryl, C₃₋₈cycloalkyl, C₃₋₈cycloalkenyl, heterocyclyl, keto(═O), —OR⁷, —C(O)R⁵, —OC(O)R⁵, S(O)ₚR⁴; ═N—O—R⁶, —NHC(O)NR⁸R⁹, —N(C₁₋₆alkyl)C(O)NR⁸R⁹, —NHC(O)R⁷, —NHCO₂R⁷, —NHSO₂(R⁴), -amidino i.e. —NHC(NH)NH₂, wherein each R′ may be optionally substituted on one or more carbon by one or more R″;
R″ at each occurrence is independently halo, azido, cyano, C₁₋₆alkyl, C₂₋₆alkenyl, C₂₋₆alkynyl, aryl, cycloalkyl, cycloalkenyl, heterocyclyl, hydroxy, —OR⁷, —C(O)R⁵, —OC(O)R⁵, S(O)ₚR⁴, —NR⁸R⁹, -amidino i.e. —NHC(NH)NH₂; provided the compound is not 8-amino-2-methoxyadenosine.

2. A compound of formula II 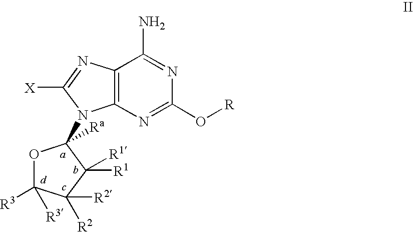
in free or salt form, wherein:
X is hydrogen, halo, NR⁸R⁹, azido, cyano, isocyano, hydroxy, C₁₋₆alkyl, C₂₋₆alkenyl, C₂₋₆alkynyl, aryl, C₃₋₈cycloalkyl, C₃₋₈cycloalkenyl, heterocyclyl, —OR⁷, —C(O)R⁵, —OC(O)R⁵, S(O)ₚR⁴, —NHC(O)NR⁸R⁹, —N(C₁₋₆alkyl)C(O)NR⁸R⁹, —NHC(O)R⁷, —NHCO₂R⁷, —NHSO₂(R⁴), -amidino i.e. —NHC(NH)NH₂; provided that when X is hydrogen, at least one carbon selected from C-a, C-b, C-c, and C-d is quaternary;
R is selected from C₁₋₈alkyl, C₂₋₈alkenyl, C₂₋₈alkynyl, C₃₋₈carbocyclyl, aryl, and heterocyclyl, any of which may be optionally substituted on one or more carbon atom by R′;
p is independently at each occurrence 0, 1 or 2;
R^{a} is hydrogen, C₁₋₆alkyl, C₂₋₆alkenyl, hydroxy(C₁₋₆alkyl), and cyano, any of which may be optionally substituted on one or more carbon atom by R′;
R¹, R^{1′}, R², R^{2′}, R³, and R^{3′} are each independently selected from hydrogen, hydroxy, cyano, azido, C₁₋₆alkyl, C₃₋₈carbocyclyl, halo, —C(O)R⁵, —OC(O)R⁵, S(O)ₚR⁴, C₂₋₆alkenyl, C₂₋₆alkynyl, heterocyclyl, —OR⁷, NR⁸R⁹, wherein R¹, R^{1′}, R², R^{2′}, R³, and R^{3′} may be optionally substituted on one or more carbon atoms by one or more R′; or alternatively,
R¹ and R^{1′}, R² and R^{2′}, or R³ and R^{3′}, taken together with the carbon to which they are attached, form C═O or C═N—O—R⁶, or an optionally substituted 3, 4, 5, 6, or 7-membered ring containing 0, 1, or 2 heteroatoms selected from O, S, NH, or N(C₁₋₆alkyl); or alternatively,
R¹ and R², R² and R³, taken together with the carbons to which they are attached, form an optionally substituted 3, 4, 5, or 6-membered ring containing 0, 1, or 2 heteroatoms selected from O, S, NH, or N(C₁₋₆alkyl);
R⁴ at each occurrence is independently —NR⁸R⁹, C₁₋₆alkyl, C₂₋₆alkenyl, C₁₋₆alkoxy, C₃₋₈cycloalkyl, heterocyclyl, and aryl wherein R⁴ may be optionally substituted on one or more carbon atoms by one or more R′;
R⁵ at each occurrence is independently hydrogen, —NR⁸R⁹, —OR⁷, C₁₋₆alkyl, C₂₋₆alkenyl, C₃₋₈cycloalkyl, C₃₋₈cycloalkenyl, heterocyclyl, and aryl wherein each R⁵ may be optionally substituted on one or more carbon atoms by one or more R′;
R⁶ at each occurrence is independently hydrogen, C₁₋₆alkyl, C₂₋₆alkenyl, C₂₋₆alkynyl, C₃₋₈cycloalkyl, C₃₋₈cycloalkenyl, heterocyclyl and aryl, wherein each R⁶ may be optionally substituted on one or more carbon atoms by one or more R′;
R⁷ at each occurrence is independently hydrogen, C₁₋₆alkyl, C₂₋₆alkenyl, C₃₋₈cycloalkyl, C₃₋₈cycloalkenyl, aryl, S(O)ₚR⁴, and heterocyclyl wherein R⁷ may be optionally substituted on one or more carbon by one or more R′;
R⁸ and R⁹ are each independently selected from hydrogen, C₁₋₆alkyl, C₂₋₆alkenyl, C₂₋₆alkynyl, —OR⁷, C₃₋₈cycloalkyl, C₃₋₈cycloalkenyl, heterocyclyl, and aryl, wherein each R⁸ or R⁹ may be optionally substituted on one or more carbon atoms by one or more R′;
R′ at each occurrence is independently halo, hydroxy, nitro, —NR⁸R⁹, azido, cyano, isocyano, C₁₋₆alkyl, C₂₋₆alkenyl, C₂₋₆alkynyl, aryl, C₃₋₈cycloalkyl, C₃₋₈cycloalkenyl, heterocyclyl, keto(═O), —OR⁷, —C(O)R⁵, —OC(O)R⁵, S(O)ₚR⁴; ═N—O—R⁶, —NHC(O)NR⁸R⁹, —N(C₁₋₆alkyl)C(O)NR⁸R⁹, —NHC(O)R⁷, —NHCO₂R⁷, —NHSO₂(R⁴), -amidino i.e. —NHC(NH)NH₂, wherein each R′ may be optionally substituted on one or more carbon by one or more R″;
R″ at each occurrence is independently halo, azido, cyano, C₁₋₆alkyl, C₂₋₆alkenyl, C₂₋₆alkynyl, aryl, cycloalkyl, cycloalkenyl, heterocyclyl, hydroxy, —OR⁷, —C(O)R⁵, —OC(O)R⁵, S(O)ₚR⁴, —NR⁸R⁹, -amidino i.e. —NHC(NH)NH₂; provided the compound is not 8-amino-2-methoxyadenosine.

3. The compound according to claim 1, which compound is selected from any one of the following: 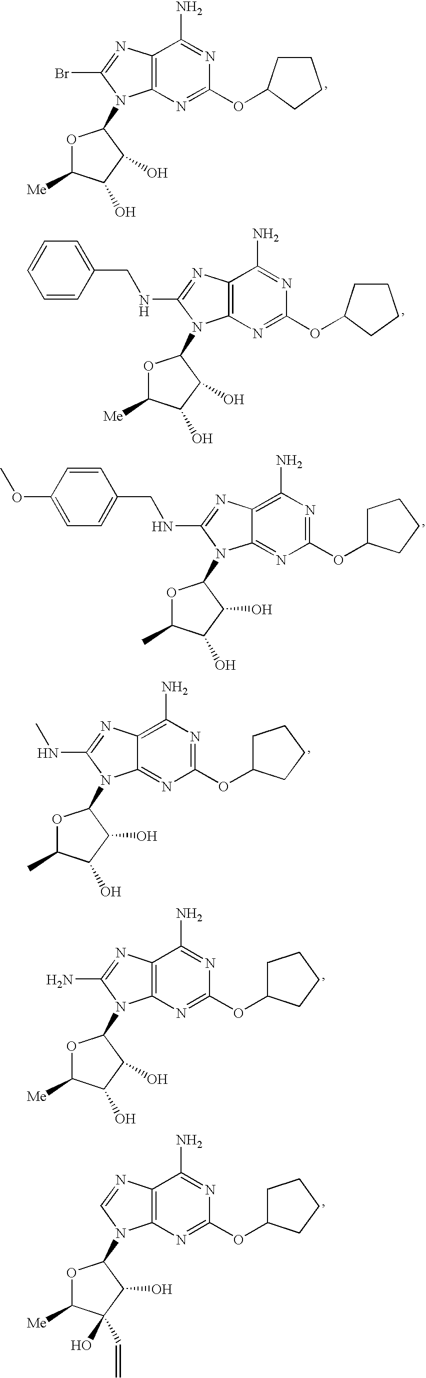 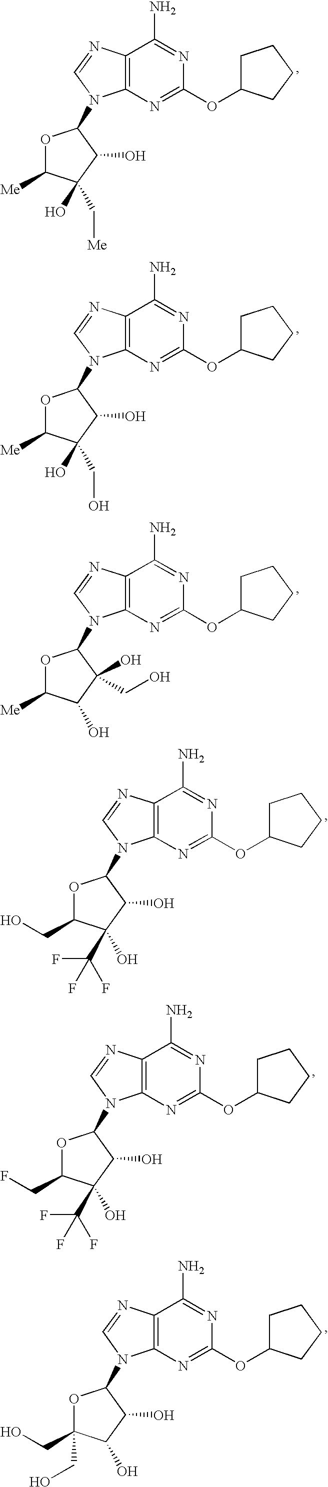 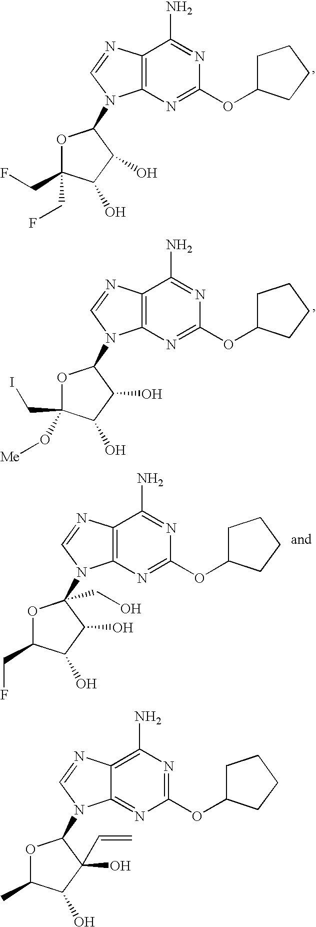
in free or salt form.

4. The compound according to claim 1 or 2, which compound is selected from a group consisting of 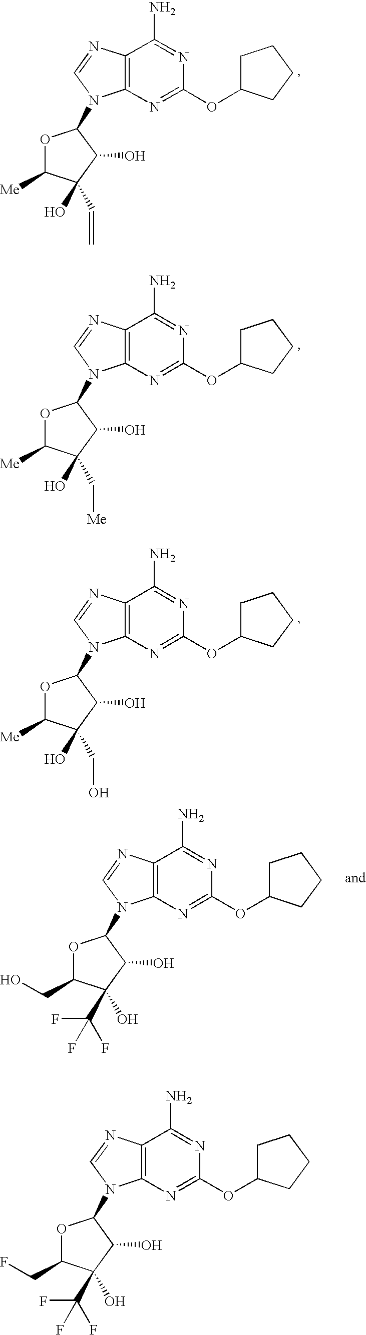
in free or salt form.

5. The compound according to claim 1 or 2, which compound is 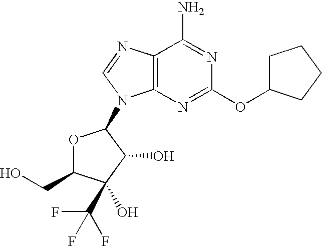
in free or salt form.

6. A method for:
i. producing an antibacterial effect,
ii. inhibition of bacterial DNA ligase, or
iii. treating a bacterial infection
in a warm blooded animal in need of such treatment, comprising administering to said animal an effective amount of a compound according to claim 1, in free or pharmaceutically acceptable salt form.

7. (canceled)

8. (canceled)

9. (canceled)

10. (canceled)

11. A pharmaceutical formulation comprising a compound according to any of the preceding claims claim 1, in free or pharmaceutically acceptable salt form, and a pharmaceutically acceptable diluent or carrier.

12. (canceled)

13. A compound of formula A 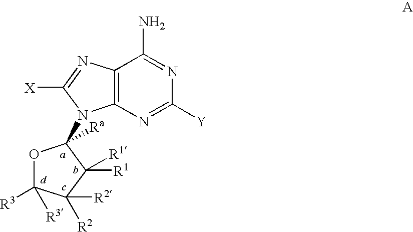
in free or salt form, wherein:
X is halo, NR⁸R⁹, azido, cyano, isocyano, hydroxy, C₁₋₆alkyl, C₂₋₆alkenyl, C₂₋₆alkynyl, aryl, C₃₋₈cycloalkyl, C₃₋₈cycloalkenyl, heterocyclyl, —OR⁷, —C(O)R⁵, —OC(O)R⁵, S(O)ₚR⁴, —NHC(O)NR⁸R⁹, —N(C₁₋₆alkyl)C(O)NR⁸R⁹, —NHC(O)R⁷, —NHCO₂R⁷, —NHSO₂(R⁴), -amidino i.e. —NHC(NH)NH₂; provided that when X is hydrogen, at least one carbon selected from C-a, C-b, C-c, and C-d is quaternary;
Y is a leaving group, e.g., halo or SO₂alkyl (e.g., SO₂Me);
p is independently at each occurrence 0, 1 or 2;
R^{a} is hydrogen, C₁₋₆alkyl, C₂₋₆alkenyl, hydroxy(C₁₋₆alkyl), and cyano, any of which may be optionally substituted on one or more carbon atom by R′;
R¹, R^{1′}, R², R^{2′}, R³, and R^{3′} are each independently selected from hydrogen, hydroxy, cyano, azido, C₁₋₆alkyl, C₃₋₈carbocyclyl, halo, —C(O)R⁵, —OC(O)R⁵, S(O)ₚR⁴, C₂₋₆alkenyl, C₂₋₆alkynyl, heterocyclyl, —OR⁷, NR⁸R⁹, wherein R¹, R^{1′}, R², R^{2′}, R³, and R^{3′} may be optionally substituted on one or more carbon atoms by one or more R; or alternatively,
R¹ and R^{1′}, R² and R^{2′}, or R³ and R^{3′}, taken together with the carbon to which they are attached, form C═O or C═N—O—R⁶, or an optionally substituted 3, 4, 5, 6, or 7-membered ring containing 0, 1, or 2 heteroatoms selected from O, S, NH, or N(C₁₋₆alkyl); or alternatively,
R¹ and R², R² and R³, taken together with the carbons to which they are attached, form an optionally substituted 3, 4, 5, or 6-membered ring containing 0, 1, or 2 heteroatoms selected from O, S, NH, or N(C₁₋₆alkyl);
R⁴ at each occurrence is independently —NR⁸R⁹, C₁₋₆alkyl, C₂₋₆alkenyl, C₁₋₆alkoxy, C₃₋₈cycloalkyl, heterocyclyl, and aryl wherein R⁴ may be optionally substituted on one or more carbon atoms by one or more R′;
R⁵ at each occurrence is independently hydrogen, —NR⁸R⁹, —OR⁷, C₁₋₆alkyl, C₂₋₆alkenyl, C₃₋₈cycloalkyl, C₃₋₈cycloalkenyl, heterocyclyl, and aryl wherein each R⁵ may be optionally substituted on one or more carbon atoms by one or more R′;
R⁶ at each occurrence is independently hydrogen, C₁₋₆alkyl, C₂₋₆alkenyl, C₂₋₆alkynyl, C₃₋₈cycloalkyl, C₃₋₈cycloalkenyl, heterocyclyl and aryl, wherein each R⁶ may be optionally substituted on one or more carbon atoms by one or more R′;
R⁷ at each occurrence is independently hydrogen, C₁₋₆alkyl, C₂₋₆alkenyl, C₃₋₈cycloalkyl, C₃₋₈cycloalkenyl, aryl, S(O)ₚR⁴, and heterocyclyl wherein R⁷ may be optionally substituted on one or more carbon by one or more R′;
R⁸ and R⁹ are each independently selected from hydrogen, C₁₋₆alkyl, C₂₋₆alkenyl, C₂₋₆alkynyl, —OR⁷, C₃₋₈cycloalkyl, C₃₋₈cycloalkenyl, heterocyclyl, and aryl, wherein each R⁸ or R⁹ may be optionally substituted on one or more carbon atoms by one or more R′;
R′ at each occurrence is independently halo, hydroxy, nitro, —NR⁸R⁹, azido, cyano, isocyano, C₁₋₆alkyl, C₂₋₆alkenyl, C₂₋₆alkynyl, aryl, C₃₋₈cycloalkyl, C₃₋₈cycloalkenyl, heterocyclyl, keto(═O), —OR⁷, —C(O)R⁵, —OC(O)R⁵, S(O)ₚR⁴; ═N—O—R⁶, —NHC(O)NR⁸R⁹, —N(C₁₋₆alkyl)C(O)NR⁸R⁹, —NHC(O)R⁷, —NHCO₂R⁷, —NHSO₂(R⁴), -amidino i.e. —NHC(NH)NH₂, wherein each R′ may be optionally substituted on one or more carbon by one or more R″;
R″ at each occurrence is independently halo, azido, cyano, C₁₋₆alkyl, C₂₋₆alkenyl, C₂₋₆alkynyl, aryl, cycloalkyl, cycloalkenyl, heterocyclyl, hydroxy, —OR⁷, —C(O)R⁵, —OC(O)R⁵, S(O)ₚR⁴, —NR⁸R⁹, -amidino i.e. —NHC(NH)NH₂.

14. The compound according to claim 13, which is 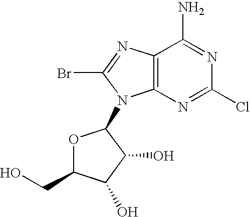
in free or salt form.

15. (canceled)

16. The compound according to claim 2, which compound is selected from any one of the following: 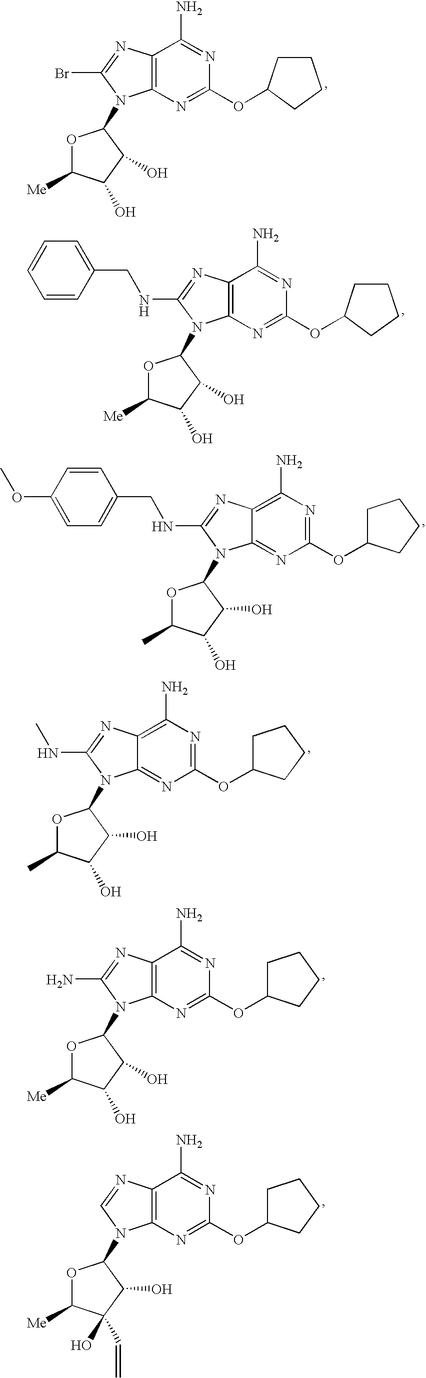 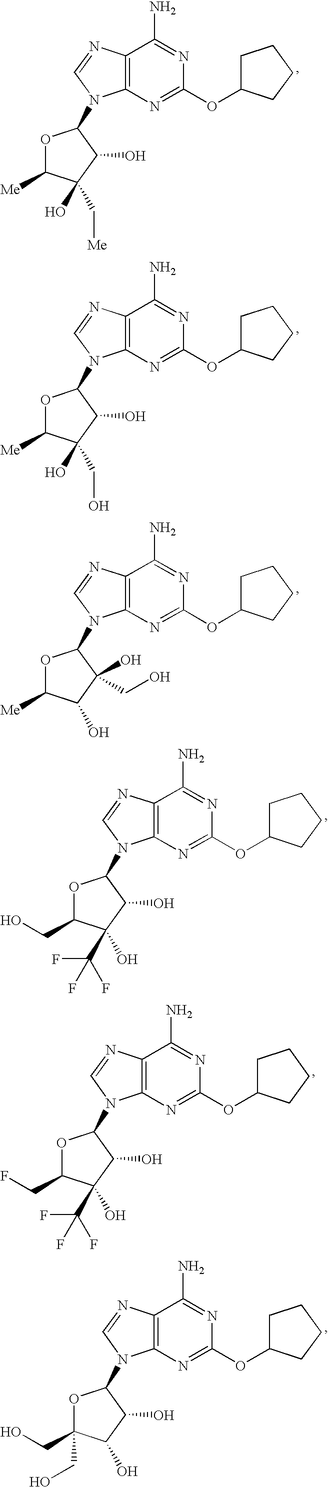 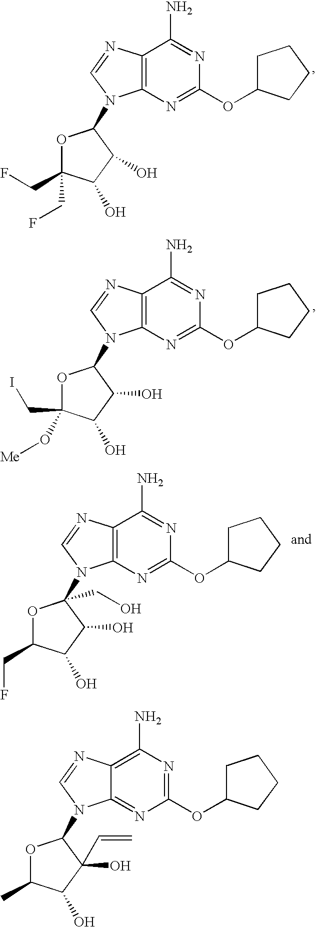
in free or salt form.

17. The compound according to claim 2, which compound is selected from a group consisting of 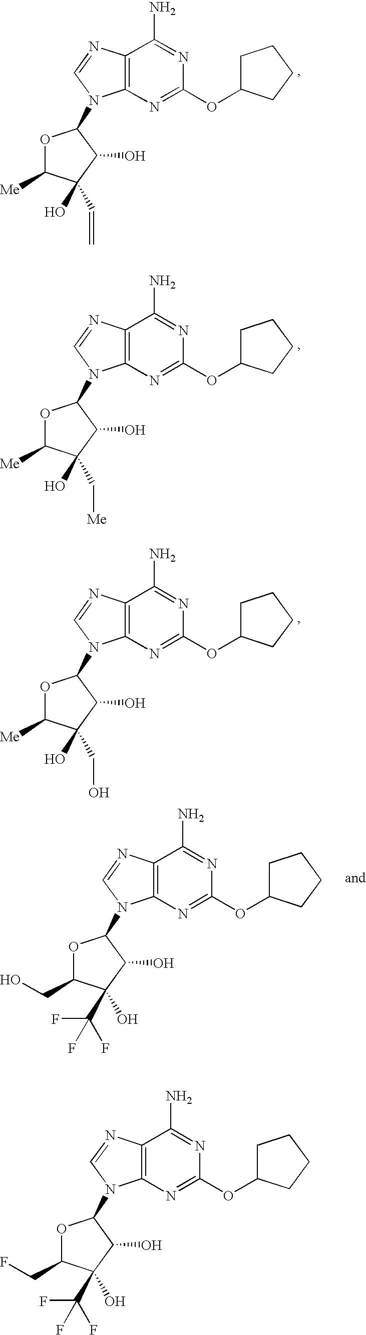
in free or salt form.

18. The compound according to claim 2, which compound is 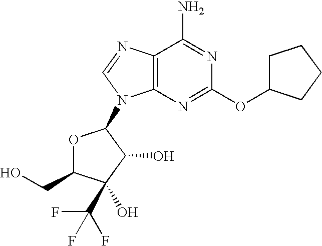
in free or salt form.

19. A method for:
i. producing an antibacterial effect,
ii. inhibition of bacterial DNA ligase, or
iii. treating a bacterial infection
in a warm blooded animal in need of such treatment, comprising administering to said animal an effective amount of a compound according to claim 2, in free or pharmaceutically acceptable salt form.
